(19) United States
(12) Patent Application Publication
(10) Pub. No.: US 2023/0270546 A1
(43) Pub. Date: Aug. 31, 2023

(54) SELF-EXPANDING, TWO COMPONENT SHEATH AND METHODS OF USING THE SAME

(71) Applicant: EDWARDS LIFESCIENCES CORPORATION, Irvine, CA (US)

(72) Inventors: Nasser William Saleh, Stockton, CA (US); Salvatore Manzella, JR., Aliso Viejo, CA (US); Roy Shitrit, Zichron Yaakov (IL)

(21) Appl. No.: 18/142,668

(22) Filed: May 3, 2023

Related U.S. Application Data
(63) Continuation of application No. PCT/US2021/058247, filed on Nov. 5, 2021.
(60) Provisional application No. 63/110,112, filed on Nov. 5, 2020.

(51) Int. Cl. A61F 2/24; A61M 25/00

(52) U.S. Cl. CPC A61F 2/2433; A61M 25/0029; A61M 25/0045; A61M 2025/0024

(57) ABSTRACT

The expandable sheaths disclosed herein include an expandable outer layer and a generally non-expandable inner layer. The outer layer is movable between a non-expanded configuration and an expanded configuration, where the outer layer/tubular layer is biased to the non-expanded configuration. Receipt of the inner layer within the central lumen of the outer layer causes the outer layer to transition from the non-expanded configuration to the expanded configuration.

### CROSS-REFERENCE TO RELATED APPLICATIONS

This application is a continuation of International Application No. PCT/US2021/058247, filed Nov. 5, 2021, which claims the benefit of U.S. Provisional Application No. 63/110,112, filed Nov. 5, 2020, the contents of which are incorporated herein by reference in its entirety.

### FIELD

The present application concerns embodiments of a sheath for use with catheter-based technologies to introduce a prosthetic device, such as a heart valve or other implant, into the patient's vasculature.

### BACKGROUND

Endovascular delivery catheter assemblies are used to implant prosthetic devices, such as a prosthetic heart valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques, including transcatheter delivery methods.

An introducer sheath can be used to safely introduce a delivery apparatus into a patient's vasculature (e.g., the femoral artery). An introducer sheath generally has an elongated sleeve that is inserted into the vasculature and a housing that contains one or more sealing valves that allow a delivery apparatus to be placed in fluid communication with the vasculature with minimal blood loss. Such introducer sheaths may be radially expandable. However, such sheaths tend to have complex mechanisms, such as ratcheting mechanisms that maintain the sheath in an expanded configuration once a device with a larger diameter than the sheath's original diameter is introduced. Existing expandable sheaths can also be prone to axial elongation as a consequence of the application of longitudinal force attendant to passing a prosthetic device through the sheath. Such elongation can cause a corresponding reduction in the diameter of the sheath, increasing the force required to insert the prosthetic device through the narrowed sheath.

Accordingly, there remains a need in the art for an improved introducer sheath for endovascular systems used for implanting valves and other prosthetic devices.

### SUMMARY

Disclosed herein are low-profile expandable introducer sheaths and methods of making and using the same to achieve low and consistent push force during intravascular procedures. The sheaths are adapted to temporarily expand a portion of the sheath to allow for the passage of a delivery system for a cardiovascular device, then return to a non-expanded state after the passage of the system. The sheath includes an expandable outer layer and an inner layer where receipt of the inner layer within the central lumen of the outer layer causes the outer layer to transition from the non-expanded configuration to the expanded configuration. The sheath can also include selectively placed longitudinal rods that mediate friction between the inner and outer tubular layers, or between folds of the inner tubular layer to facilitate easy expansion and collapse. This reduces the overall push force and increases the consistency of the push force needed the advance the oversized implant through the sheath's lumen. The lower and more consistent push force facilitates a reduction in the profile size.

An expandable sheath for delivering a device to a treatment site within a patient includes an outer layer and an inner layer. The outer layer (e.g., expandable outer layer), including an elongated tubular member with integrated axial support extending longitudinally therein, the outer layer movable between a non-expanded configuration and an expanded configuration, where the outer layer/tubular layer is biased to the non-expanded configuration. The inner layer is received within a central lumen of the outer layer and movable therein. Receipt of the inner layer within the central lumen of the outer layer causes the outer layer to transition from the non-expanded configuration to the expanded configuration.

A method of delivering a device into the blood vessel of a patient using the expandable introducer sheath can include inserting an outer layer at an implantation site within a blood vessel of the patient, the outer layer including an elongated tubular member with integrated axial supports extending longitudinally therein, an introducer extending within a central lumen of the outer layer, where the outer layer movable between a non-expanded configuration and an expanded configuration, where the outer layer is biased to the non-expanded configuration; removing the introducer from the central lumen of the outer layer; advancing an inner layer within the central lumen of the outer layer, an introducer extending within a central lumen of the inner layer; locally expanding a portion of the outer layer from the non-expanded configuration towards the expanded configuration by a radially outward force exerted on an inner surface of the outer layer by the advancement of the inner layer; removing the introducer from within the central lumen of the inner layer; advancing a prosthetic delivery device through a central lumen of the inner layer; delivering a prosthetic delivery device to a treatment site; removing the prosthetic delivery device from the central lumen of the inner layer; removing the inner layer from the central lumen of the outer layer; and locally contracting the portion of the outer layer from the expanded configuration at least partially back to the non-expanded configuration upon removal of the inner layer from the portion of the outer layer.

A delivery catheter assembly for delivering a device to a treatment site within a patient includes a proximal region comprising a hub with a hemostasis valve and an expandable sheath comprising an outer layer and an inner layer where the outer layer is coupled to and extending distally from the hub and fluidically coupled to the hemostasis valve. The outer layer (e.g., expandable outer layer) includes an elongated tubular member with integrated axial support extending longitudinally therein, the outer layer movable between a non-expanded configuration and an expanded configuration, where the outer layer/tubular layer is biased to the non-expanded configuration. The inner layer is received within a central lumen of the outer layer and movable therein. Receipt of the inner layer within the central lumen of the outer layer causes the outer layer to transition from the non-expanded configuration to the expanded configuration. The delivery catheter assembly further includes a guide catheter slidably positionable within a central lumen of the inner layer; a balloon catheter positionable within the guide catheter, a distal region of the balloon catheter comprising an inflatable balloon; an implantable device configured to be coupled to the inflatable balloon, and a capsule configured to extend over the implantable device.

An expandable sheath for delivering a device to a treatment site within a patient includes an outer layer and an inner layer, where receipt of the inner layer within the central lumen of the outer layer causes the outer layer to transition from the non-expanded configuration to the expanded configuration. The outer layer (e.g., expandable outer layer) includes a first polymeric layer and a braided layer comprising a plurality of filaments braided together, the outer layer movable between a non-expanded configuration and an expanded configuration, where the outer layer/tubular layer is biased to the non-expanded configuration. The inner layer (e.g., flexible inner layer) is received within a central lumen of the outer layer and movable therein.

A method of delivering a device into the blood vessel of a patient using the expandable introducer sheath can include inserting an outer layer at an implantation site within a blood vessel of the patient, the outer layer including an a first polymeric layer and a braided layer comprising a plurality of filaments braided together, an introducer extending within a central lumen of the outer layer, where the outer layer movable between a non-expanded configuration and an expanded configuration, where the outer layer is biased to the non-expanded configuration; removing the introducer from the central lumen of the outer layer; advancing an inner layer within the central lumen of the outer layer, an introducer extending within a central lumen of the inner layer; locally expanding a portion of the outer layer from the non-expanded configuration towards the expanded configuration by a radially outward force exerted on an inner surface of the outer layer by the advancement of the inner layer; removing the introducer from within the central lumen of the outer layer; advancing a prosthetic delivery device through a central lumen of the inner layer; delivering a prosthetic device to a treatment site; removing the prosthetic delivery device from the central lumen of the inner layer; removing the inner layer from the central lumen of the outer layer; locally contracting the portion of the outer layer from the expanded configuration at least partially back to the non-expanded configuration upon removal of the inner layer from the portion of the outer layer; and removing the outer layer from patient's blood vessel.

A delivery catheter assembly for delivering a device to a treatment site within a patient includes a proximal region comprising a hub with a hemostasis valve and an expandable sheath including an outer layer and an inner layer, the outer layer coupled to and extending distally from the hub and fluidically coupled to the hemostasis valve. Receipt of the inner layer within the central lumen of the outer layer causes the outer layer to transition from the non-expanded configuration to the expanded configuration. The outer layer (e.g., expandable outer layer) includes a first polymeric layer and a braided layer comprising a plurality of filaments braided together, the outer layer movable between a non-expanded configuration and an expanded configuration, where the outer layer/tubular layer is biased to the non-expanded configuration. The inner layer (e.g., flexible inner layer) is received within a central lumen of the outer layer and movable therein. The delivery catheter assembly further comprising a guide catheter slidably positionable within a central lumen of the inner layer; a balloon catheter positionable within the guide catheter, a distal region of the balloon catheter comprising an inflatable balloon; an implantable device configured to be coupled to the inflatable balloon, and a capsule configured to extend over the implantable device.

### DESCRIPTION OF DRAWINGS

FIG. 1 is a side view of a delivery apparatus for delivering a prosthetic implant.

FIG. 2 is a side view of an expandable sheath assembly in use with the delivery apparatus of FIG. 1.

FIG. 3 is a side view of the outer layer of FIG. 2 including an introducer.

FIG. 4 is a side cross-section view of the outer layer and introducer of FIG. 3.

FIG. 5 is a side cross-section view of the outer layer of FIG. 2.

FIGS. 6A and 6B are enlarged partial cross-section views of the outer layer of FIG. 5.

FIGS. 7A and 7B are radial cross-section views of the outer layer of FIG. 2 in the non-expanded and expanded configuration.

FIGS. 8A and 8B are partial perspective views of a portion of the outer layer of FIG. 2 in the non-expanded and expanded configuration.

FIG. 9 is a side view of the inner layer of FIG. 2 including an introducer.

FIG. 10 is a side cross-section view of the inner layer and introducer of FIG. 9.

FIG. 11 is a side cross-section view of the inner layer of FIG. 9.

FIGS. 12A and 12B are enlarged partial cross-section views of the inner layer of FIG. 11.

FIG. 13 is a side view of the inner and outer layers of FIG. 2 including an introducer.

FIG. 14 is an enlarged side view of the inner and outer layers of FIG. 13.

FIG. 15 is a side view of the inner and outer layers of FIG. 2 including an introducer.

FIG. 16 is an enlarged side view of the inner and outer layers of FIG. 15.

FIG. 17 is a side cross-section view of the inner and outer layers of FIG. 2 including an introducer.

FIG. 18 is an enlarged partial cross-section view of the inner and outer layers of FIG. 17.

FIG. 19 is a side view of the inner and outer layers of FIG. 2.

FIG. 20 is an enlarged side view of the inner and outer layers of FIG. 19.

FIG. 21 is a cross-section view of the inner and outer layers of FIG. 19.

FIG. 22 is a side cross-section view of the inner and outer layers of FIG. 2

FIG. 23 is an enlarged partial cross-section view of the inner and outer layers of FIG. 22.

FIG. 24 is a side view of an expandable sheath assembly in use with the delivery apparatus of FIG. 1.

FIG. 25 is a side view of the outer layer of FIG. 24.

FIG. 26 is a side view of the outer layer of FIG. 24.

FIG. 27 is a magnified view of a portion of the expandable sheath of FIG. 24.

FIG. 28 is a side elevation cross-sectional view of a portion of the expandable sheath of FIG. 24.

FIG. 29A is a magnified view of a portion of the expandable sheath of FIG. 24 with the outer polymeric layer removed for purposes of illustration.

FIG. 29B is a magnified view of a portion of the braided layer of the sheath of FIG. 24.

FIG. 30 is a magnified view of a portion of the expandable sheath of FIG. 24 illustrating expansion of the sheath as a prosthetic device is advanced through the sheath.

FIG. 31 is a side view of the inner layer of FIG. 24.

FIG. 32 is a side view of the inner layer of FIG. 24.

FIG. 33 is a partial side view of the inner and outer layer of FIG. 24.

FIG. 34 is a side view of the inner and outer layer of FIG. 24 including an introducer.

FIG. 35 is a partial side view of the inner and outer layer of FIG. 29.

FIG. 36 is a partial side view of the inner and outer layer of FIG. 29.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or a combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the present disclosure are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The disclosure is not restricted to the details of any foregoing embodiments. This disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The terms “proximal” and “distal” as used herein refer to regions of a sheath, catheter, or delivery assembly. “Proximal” means that region closest to handle of the device, while “distal” means that region farthest away from the handle of the device.

Disclosed herein are elongate, expandable introducer sheaths that are particularly suitable for use in the delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Patent Application Publication No. 2012/0123529, both of which are hereby incorporated by reference. The elongate expandable introducer sheaths disclosed herein can also be used with the delivery systems for other types of implantable devices, such as self-expanding implantable heart valves, stents or filters. The term “implantable” as used herein is broadly defined to mean anything—prosthetic or not—that is delivered to a site within a body. A diagnostic device, for example, can be implantable.

Disclosed embodiments of an expandable introducer sheath/sheath assembly can minimize trauma to the vessel by allowing for temporary expansion of a portion of the introducer sheath to accommodate the implantable device and its delivery system, then return fully or partially to the original, non-expanded diameter after passage of the device. The expandable sheath can include an expandable outer tubular layer and a reinforced and/or flexible inner tubular layer received within the outer tubular layer. The outer tubular layer provides a relatively flexible layer with a small diameter/profile sheath. The outer tubular layer is inserted first, in a non-expanded configuration, into the patient's vasculature. Its relatively low profile and softness enable it to easily bend along the vascular path with minimal risk of trauma.

The larger diameter inner tubular layer is provided as a separate non-expandable (or only partially expandable) introducer sheath. The inner tubular layer provides higher puncture and rupture resistance, with higher rigidity or column strength than that of the expandable outer layer. The inner layer is inserted into the outer tubular layer and the larger cross-sectional diameter of the inner layer temporarily expands or stretches the outer tubular layer and provides an enlarged central lumen to allow passage of an implant. The outer tubular layer is biased towards a non-expanded state such that when the inner layer is removed, the outer tubular layer returns to/toward its non-expanded, reduced, diameter configuration.

Because the outer layer and the inner layer are inserted separately/sequentially, this allows for the initial introduction of an expandable sheath/outer layer with a smaller profile than the profiles of prior art introducer sheaths (e.g., 12 F or less). Using the increased diameter inner layer to temporarily expand the outer layer reduces/avoids the vessel trauma caused by the introduction of a larger diameter sheath and longer period of vessel expansion of prior art sheaths. Because the inner layer is not required to expand, in contrast to prior art sheaths, the radial and axial support structure provides increased kink resistance. Additionally, in providing a temporarily enlarged central lumen of the sheath assembly/inner layer along the length of the sheath, the delivery device and/or prosthetic device are no longer required to provide the radially outward force necessary to expand the sheath. Rather, the larger diameter inner layer is facilitating the expansion and maintaining an enlarged diameter delivery lumen. As a result, the shear force experienced in the vessel wall by the passing/expanding device is minimized, puncture and rupture resistance is increased, insertion forces into the artery are reduced, and the push force to move the device through the sheath is significantly reduced. This allows for the use of much smaller and/or delicate delivery devices that could not withstand the entry push force, i.e., devices that are generally not be strong enough to expand the sheath and/or vessel wall such as transcatheter coronary revascularization, transcatheter left atrial appendage closure, transcatheter septal closure devices, transcatheter ventricular assist device, and other still-in-development therapies.

The inner layer can have a higher column strength than the expandable outer layer. As a result, the assembly of both the inner and outer layers provides both the required flexibility during initial insertion into the vasculature, as well as the required column strength during retraction of the delivery system that prevents the sheath assembly from kinking or collapsing. Additionally, the flexible, more rigid inner layer, may further provide some straightening effect to the tortious vascular path, resulting in easier insertion of the delivery apparatus through the sheath assembly.

In another aspect, the expandable sheath can include one or more longitudinally oriented axial supports, such as rods. The axial supports can be provided in the inner or outer layer. The axial supports extend from a surface of the inner and/or outer layer and provide a contact surface for adjacent sheath structure. The push force is decreased because the axial supports lower the friction between the surfaces of the inner and outer layer and/or the inner layer and the passing device. As a result, the push force required for insertion of the inner layer and/or device is reduced.

The sheath assembly is versatile in that it enables the clinician to choose whether to use only the expandable, with lower profile and relatively soft outer layer, only the relatively more rigid flexible inner layer, or both according to patient-specific anatomy and/or personal preferences of the clinician.

Finally, present embodiments can reduce the length of time a procedure takes, as well as reduce the risk of a longitudinal or radial vessel tear or plaque dislodgement because only two sheath layers are required, rather than several different sheaths of gradually increasing diameters. Embodiments of the present expandable sheath can avoid the need for multiple insertions for the dilation of the vessel.

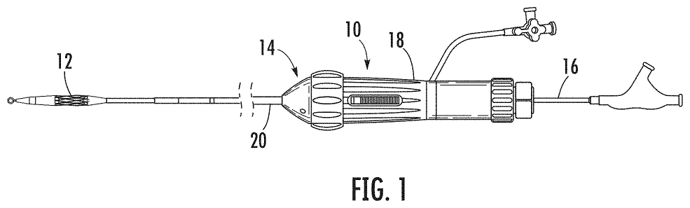

FIG. 1 illustrates a representative delivery apparatus 10 for delivering a medical device 12, such as a prosthetic heart valve or other prosthetic implant, to a patient. The delivery apparatus 10 is exemplary only and can be used in combination with any of the expandable sheath embodiments described herein. Likewise, the sheaths described herein ca be used in combination with any of various known delivery apparatuses. The delivery apparatus 10 generally includes a steerable guide catheter 14 and a balloon catheter 16 extending through the guide catheter 14. A prosthetic device, such as a prosthetic heart valve 12, can be positioned on the distal end of the balloon catheter 16. The guide catheter 14 and the balloon catheter 16 can be adapted to slide longitudinally relative to each other to facilitate delivery and positioning of a prosthetic heart valve 12 at an implantation site in a patient's body, as described in more detail below. The guide catheter 14 includes a handle 18 and an elongated guide tube or shaft 20 extending from the handle 18. Additional examples of introducer devices and expandable sheaths can be found in U.S. Patent Publication No. 2019/0307589, entitled “Expandable Sheath,” and U.S. Provisional Patent Application No. 62/912,569, entitled “Expandable Sheath,” which are incorporated by reference in their entireties.

The prosthetic heart valve 12 can be delivered into a patient's body in a radially compressed configuration and radially expanded to a radially expanded configuration at the desired deployment site. In the illustrated embodiment, the prosthetic heart valve 12 is a plastically expandable prosthetic valve that is delivered into the patient's body in a radially compressed configuration on a balloon of the balloon catheter 16 (as shown in FIG. 1) and then radially expanded to a radially expanded configuration at the deployment site by inflating the balloon (or by actuating another type of expansion device of the delivery apparatus). Further details regarding a plastically expandable heart valve that can be implanted using the devices disclosed herein are disclosed in U.S. Publication No. 2012/0123529, entitled “Prosthetic Heart Valve,” which is incorporated herein by reference. In other embodiments, the prosthetic heart valve 12 can be a self-expandable heart valve that is restrained in a radially compressed configuration by a sheath or other component of the delivery apparatus (e.g., a delivery capsule) and self-expands to a radially expanded configuration when released by the sheath or other component of the delivery apparatus. Further details regarding a self-expandable heart valve that can be implanted using the devices disclosed herein are disclosed in U.S. Publication No. 2012/0239142, entitled “Prosthetic Heart Valve Delivery Apparatus,” which is incorporated herein by reference. In still other embodiments, the prosthetic heart valve 12 can be a mechanically expandable heart valve that comprises a plurality of struts connected by hinges or pivot joints and is expandable from a radially compressed configuration to a radially expanded configuration by actuating an expansion mechanism that applies an expansion force to the prosthetic valve. Further details regarding a mechanically expandable heart valve that can be implanted using the devices disclosed herein are disclosed in U.S. Publication No. 2018/0153689, entitled “Mechanically Expanding Heart Valve and Delivery Apparatus,” which is incorporated herein by reference. In still other embodiments, a prosthetic valve can incorporate two or more of the above-described technologies. For example, a self-expandable heart valve can be used in combination with an expansion device to assist expansion of the self-expandable heart valve.

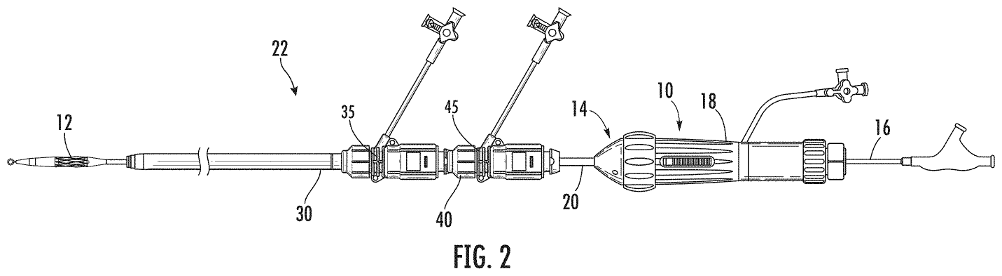

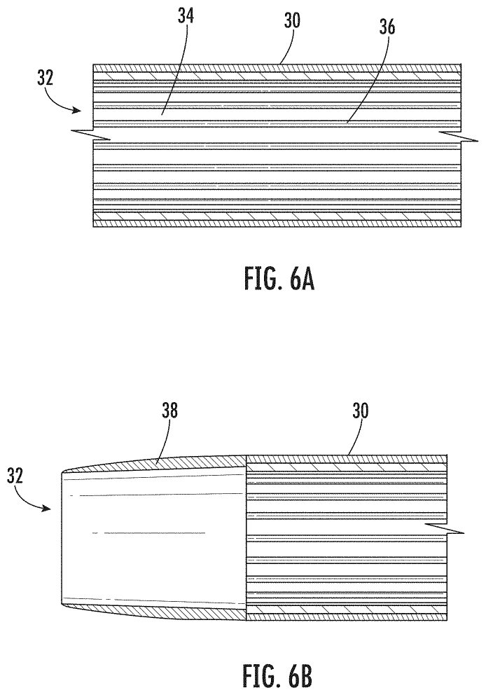

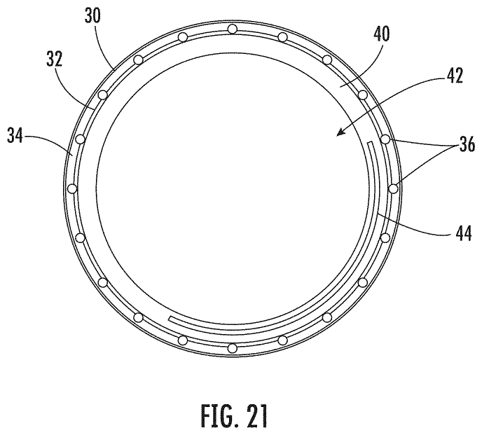

FIG. 2 illustrates a first example expandable sheath assembly 22 (which can be referred to as an introducer sheath device or assembly) that can be used to introduce the delivery apparatus 10 and the prosthetic heart valve 12 into a patient's body. The expandable sheath assembly 22 includes an outer tubular layer 30 (also referred to herein as outer layer and expandable outer layer) and an inner tubular layer 40 (also referred to herein as inner layer, reinforced layer) received within a central lumen 32 (FIG. 6A) of the outer layer 30 and movable therein. The expandable sheath assembly 22, and in particular the inner layer 40, has a central lumen 42 (FIG. 21) to guide passage of the delivery apparatus 10.

Generally, during use a distal end of the outer layer 30 is passed through the skin of the patient and is inserted into a vessel, such as the femoral artery. As will be described in more detail below, the outer layer 30 provides a small diameter/profile sheath that can be inserted into the patient's vasculature with minimal risk of trauma. The inner layer 40, having a larger diameter than outer layer 30, is inserted into the central lumen 32 of the outer layer 30 temporarily expanding the outer layer 30 from the non-expanded configuration to an expanded configuration, and providing an enlarged central lumen 42 to allow passage of the delivery apparatus 10 and the prosthetic heart valve 12. The enlarged diameter of the central lumen 42 of the inner layer 40 decreases the required push force to move the prosthetic heart valve 12 through the sheath assembly 22.

The delivery apparatus 10, with the prosthetic heart valve 12 mounted thereon, are then inserted through the hub(s) and the inner layer 40, and advanced through the patient's vasculature to the treatment site, where the implant is to be delivered and implanted within the patient.

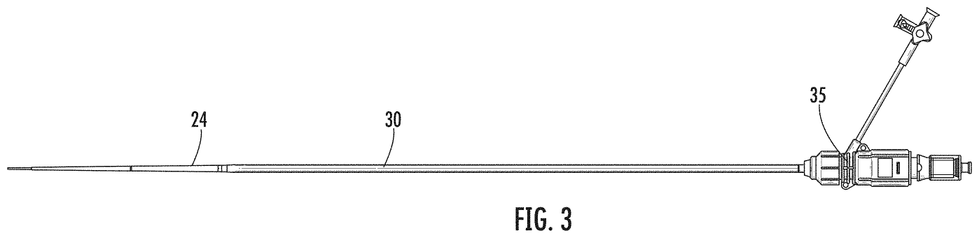

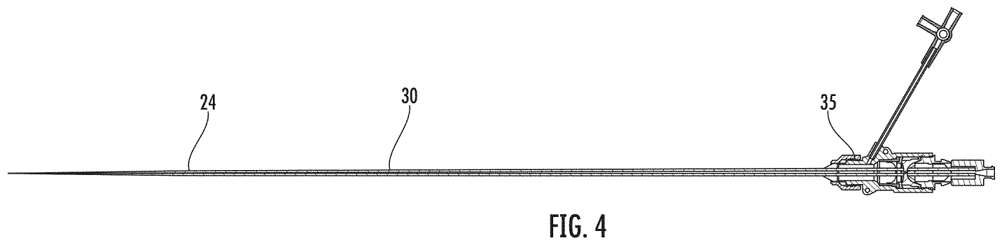

FIG. 3 provides a side view of the outer layer 30 of FIG. 2 including an introducer 24. FIG. 4 provides a side cross-section view of the outer layer 30 and introducer 24. As described above, the outer layer 30 provides a relatively flexible layer with a small diameter/profile in non-expanded configuration. During insertion, the introducer 24 is provided within the central lumen of the outer layer 30 to maintain the shape of the sheath and provide the necessary column strength during insertion, helping prevent the outer layer 30 from kinking and/or collapsing. The outer layer 30 protects vasculature, prevent calcification dislodgement, and transfers the potentially traumatic axial force of insertion to safe, radial force on the vasculature. Example expandable outer layer 30/expandable introducer sheaths are disclosed in commonly assigned U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. application Ser. No. 14/880,109, entitled “Expandable Sheath,” U.S. application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. application Ser. No. 15/997,587, entitled “Expandable Sheath for Introducing an Endovascular Delivery Device into a Body,” U.S. application Ser. No. 16/378,417, entitled “Expandable Sheath,” the disclosures of which are herein incorporated by reference.

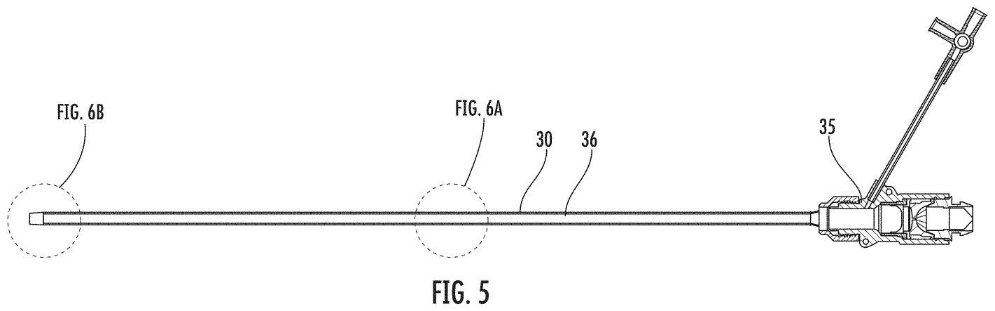

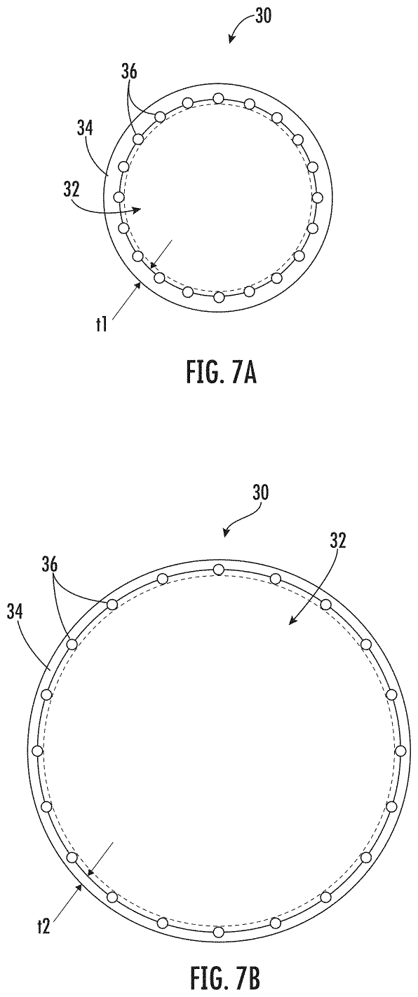

The outer layer 30 is defined by an elongated tubular member 34 with an integrated axial support 36 extending longitudinally therein. FIG. 5 provides a side cross-section view of the outer layer 30 without the introducer 24. FIGS. 6A and 6B are enlarged side cross-section views of the outer layer 30 and FIGS. 7A and 7B are radial cross-section views of the outer layer 30. As illustrated in FIGS. 5-7B, the outer layer 30 can include a plurality of axial supports 36 spaced circumferentially around elongated tubular member 34. As provided in FIGS. 7A and 7B, each of the plurality of axial supports 36 can be equally spaced and/or symmetrically spaced circumferentially around the elongated tubular member 34.

As described above, the outer layer 30 is movable between a non-expanded configuration and an expanded configuration, where the diameter of the outer layer 30 is greater in the expanded configuration. In some examples, the inner diameter of the central lumen 32 of the outer layer 30 in the non-expanded configuration ranges between about 8 F and about 14 F, and the inner diameter of the central lumen 32 in the expanded configuration ranges between about 14 F and about 28 F. In some examples, the inner diameter of the central lumen 32 is about 24 F in the expanded configuration.

In general, the outer layer 30 is biased to the non-expanded configuration such that absent any radially directed force driving expansion, the outer layer 30 will return to/toward its non-expanded configuration.

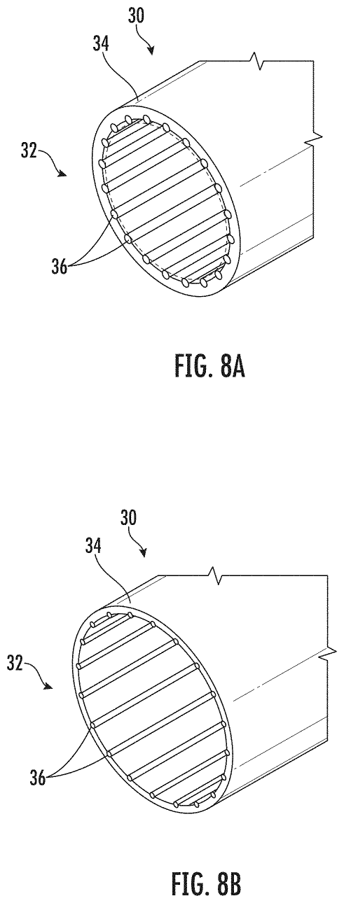

FIG. 7A provides a radial cross-section view of the outer layer 30 in a non-expanded configuration, and FIG. 7B provides a radial cross-section view of the outer layer 30 in an expanded configuration. Similarly, FIG. 8A provides a partial perspective view of a portion of the outer layer 30 in a non-expanded configuration, and FIG. 8B provides a partial perspective view of a portion of the outer layer 30 in an expanded configuration. As illustrated in FIGS. 7B and 8B, when the outer layer 30 transitions from the non-expanded configuration to the expanded configuration, the circumferential spacing between each of the plurality of axial supports 36 increases. Likewise, when the outer layer 30 transitions from the expanded configuration to the non-expanded configuration, the circumferential spacing between each of the plurality of axial supports 36 decreases. As illustrated in in FIGS. 7B and 8B, when the outer layer 30 transitions from the non-expanded configuration to the expanded configuration, the wall thickness of the tubular member 34 decreases from t₁ to t₂, where t₁ is greater than t₂.

It is contemplated that the number of axial supports 36 can range between 2 and 30 individual axial supports 36. For example, the outer layer provided in FIGS. 7A and 7B includes 20 individual axial supports 36.

As illustrated in FIGS. 6A-7B, a portion of an outer surface of the axial supports 36 protrudes from an inner surface of the central lumen 32 of the outer layer 30. As such that outer surface of the axial supports 36 facilitates relative movement between the outer layer 30 and the inner layer 40 as it moves within the central lumen 32 of the outer layer 30. The axial supports 36 reduce contact area between the outer layer 30 and the inner layer 40, thereby reducing friction between the two.

As provided in FIG. 5, the axial supports 36 extend along an entire length of the outer layer 30. However, it is contemplated that the axial supports 36 can extend along only a portion of the entire length of the outer layer 30.

The axial supports 36 may have a curvilinear, rectilinear, and irregular shape in cross-section, or combinations thereof. As illustrated in FIGS. 7A and 7B, the axial supports 36 have a generally circular shape in cross-section. In some examples, the diameter of the axial supports 36 ranges between about 0.020 inches and about 0.005 inches. In some examples, the diameter of the axial supports is about 0.010 inches. Though the axial supports of FIGS. 7A-7B are illustrated with consistent size and cross-sectional shape, it is contemplated that the size and/or shape of the individual axial supports 36 may vary around the circumference of the outer layer 30.

In general, the outer layer 30 is highly flexible allowing it to conform to the patient anatomy as it tracks trough femoral vasculature. The soft material of the tubular member 34 allows the sheath to expand radially while the stiffer axial supports 36 provide necessary column strength. For example, the tubular member 34 is composed of an elastic material that easily stretches between the non-expanded and the expanded configuration, while the axial supports 36 are composed of a more rigid/stiffer material with a higher column strength than the tubular member 34. As such, the axial supports 36 provide column strength to the outer layer 30 that help to prevent the sheath from kinking or collapsing during insertion and delivery/retrieval of the prosthetic device. In some examples, the column strength of the axial supports 36 is also greater than the column strength of the inner layer 40.

In some examples, the tubular member 34 is composed of an elastomeric material. Example materials include a styrene-based elastomer, polyurethane, latex, copolymers thereof, blends thereof, or co-extrudates of thereof. In general, the tubular member 34 is composed of a material having a high stretching ratio. For example, the tubular member 34 can have a 2:1 stretching ratio such that the tubular member 34 is composed of a material that has a stretched percentage of 100% unstretched compared to stretched. In contrast, the axial supports 36 are composed of a material with a higher durometer than the elongated tubular member 34. For example, the durometer of the axial supports 36 ranges between about 10 D and about 75 D, and the durometer of the tubular member 34 ranges between about 10 A and about 45 D.

To prevent the outer layer 30 from lengthening/constricting during delivery of the prosthetic device, it is necessary for the axial supports 36 to be composed of a material that experiences minimal stretching/elongation when under tension. In some examples, the axial supports 36 are composed of a material that stretches/elongates less than 1% under tension. In other examples, the axial supports 36 are composed of a material that does not stretch/elongate under tension. Accordingly, the overall length of the outer layer 30 does not change when the outer layer 30 transitions between the non-expanded and the expanded configuration.

In some examples, the axial supports 36 are composed of a material comprising a high-density polyethylene, polypropylene, polyamide, fluoropolymer, copolymers thereof, or blends thereof. In further examples, the axial supports 36 are composed of a material comprising a metal, a shape memory alloy, or a combination thereof. For example, the axial supports 36 can be composed of a shape memory alloy such as nitinol. In an example system, the axial supports 36 are composed of a coiled wire proving increased flexibility to the expandable outer layer. It is contemplated that each of the plurality of axial supports 36 can be composed of the same or a different material. The axial supports 36 can be integrally formed with the outer layer 30 (e.g., co-extruded). In other examples, the axial supports 36 are separately formed from the outer layer 30 and the outer layer 30 is provided over the axial supports 36 (e.g., the outer layer 30 is extruded over the axial supports 36).

While the outer surface of the tubular member 34/outer layer 30 includes has a generally constant cylindrical outer diameter, the distal end of the outer layer 30 can include an atraumatic, expandable distal tip 38. As illustrated in FIGS. 5 and 6B, the distal tip 38 defines a tubular structure with a slightly tapering or frusto-conical distal end. The outer surface of the distal tip 38 includes a distally tapering and/or curved shape such that a diameter of the distal tip 38 adjacent the tubular member 34 is greater than the diameter of the distal end of the distal tip 38. In some examples, the inner surface of the central lumen of the distal tip 38 includes a distally tapering and/or curved shape. In other examples, the central lumen of the distal tip 38 has a constant inner diameter. As illustrated in FIG. 6B, the thickness of the distal tip 38 decreases between the proximal and distal ends. The distal tip 38 is formed of an elastomeric material that expands with the tubular member 34 between the non-expanded and expanded configurations. In some examples, the distal tip 38 is formed from the same material as the tubular member. In other examples, the distal tip 38 is formed from a different elastic material as the tubular member. In some examples, the distal tip 38 is integrally formed with the tubular member 34. For example, the distal tip 38 can be co-extruded with the tubular member 34 or reflowed from the existing tubular member 34 into the desired shape. In other examples, the distal tip 38 is separately formed and coupled to the tubular member 34. For example, the distal tip 38 can be extruded/molded separately and coupled to the tubular member 34 using heat processing.

The structure of the distal tip 38 of the outer layer 30 helps to increase the structural rigidity of the distal end of the tubular wall structure, blocks blood flow between the layers and provides a smooth, tapered profile for pushing through tissue when advanced over a wire or dilator.

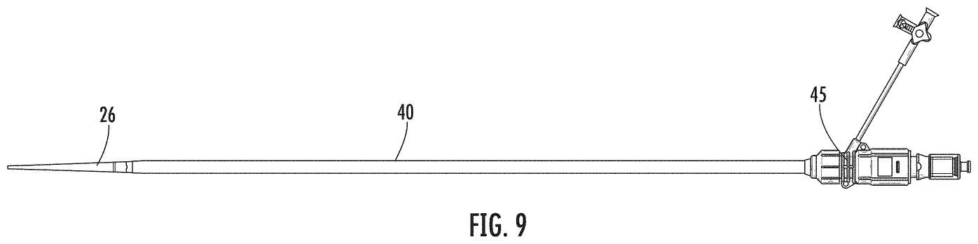

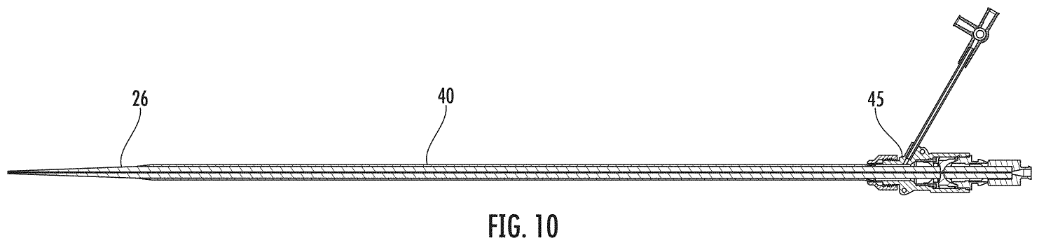

As described above, the expandable sheath assembly 22 includes an inner layer 40. FIG. 9 provides a side view of the inner layer 40 of FIG. 2 including an introducer 26. FIG. 10 provides a side cross-section view of the inner layer 40 and introducer 26. As described above, when the inner layer 40 is inserted into the central lumen 32 of the outer layer 30, it causes the outer layer 30 to locally expand from the non-expanded configuration to the expanded configuration by the radially outward force exerted on the surface of the central lumen 32 of the outer layer 30 by the inner layer 40. The enlarged diameter of the central lumen 42 of the inner layer 40 is large enough that the delivery apparatus 10/prosthetic heart valve 12 can pass through without additional expansion of the outer layer 30 and/or the inner layer 40, decreasing the required push force to move the device through the sheath assembly 22. As the inner layer 40 is removed from the outer layer 30, the outer layer 30 locally contracts from the expanded configuration at least partially back to the non-expanded configuration.

In general, the inner layer 40 does not expand radially or experiences minimal radial expansion. In some examples, the radial expansion of the inner layer 40 is less than 1%. The inner diameter of the central lumen 42 of the inner layer 40 ranges between about 16 F and about 28 F. In some examples, the inner diameter ranges between about 20 F and about 28 F. In use, the clinician can select an inner layer having a diameter based on the particular therapy and size of the delivery apparatus 10. Generally, the diameter is selected as one greater than the outer diameter of the delivery apparatus 10 to minimize trauma and unnecessary expansion, but large enough that the delivery apparatus 10 and prosthetic heart valve 12 can pass freely therethrough.

As described above, the inner layer 40 provides a more flexible and rigid inner layer to the expandable sheath assembly 22 when compared to the outer layer 30. Because the inner layer 40 is more rigid than the outer layer 30 it will have better kink resistance upon axial loading (e.g., during insertion and retrieval of the delivery apparatus 10/prosthetic heart 12). Additionally, the inner layer 40 will straighten, to some degree, the anatomy/vascular path, facilitating easier insertion of the delivery apparatus 10 through the expandable sheath assembly 22. For example, the inner layer 40 can be composed of a stiff material. In some examples, the inner layer 40 has a column strength equal to or greater than a column strength of the outer layer 30. The inner layer 40 can have a durometer ranging between about 25 D and about 75 D. In some examples, the durometer varies along the entire length of the inner layer 40. It is also contemplated that the durometer can be consistent along the entire length of the inner layer 40.

In certain examples, the inner layer 40 can comprise a lubricious, low-friction, and/or relatively non-elastic material. Exemplary materials can include ultra-high-molecular-weight polyethylene (UHMWPE) (e.g., Dyneema®), high-molecular-weight polyethylene (HMWPE), high density polyethylene (HDPE), or polyether ether ketone (PEEK). With regard to the inner layer 40 in particular, the use of low coefficient of friction materials can facilitate passage of the prosthetic device through the central lumen 42. Other suitable materials for the inner layer 40 can include polytetrafluoroethylene (PTFE), polyethylene terephthalate (PET), expanded polytetrafluoroethylene (ePTFE), ethylene tetrafluoroethylene (ETFE), nylon, polyethylene, polyether block amide (e.g., Pebax), nylon, fluorinated ethylene propylene (FEP), polypropylene (PP), and/or combinations of any of the above.

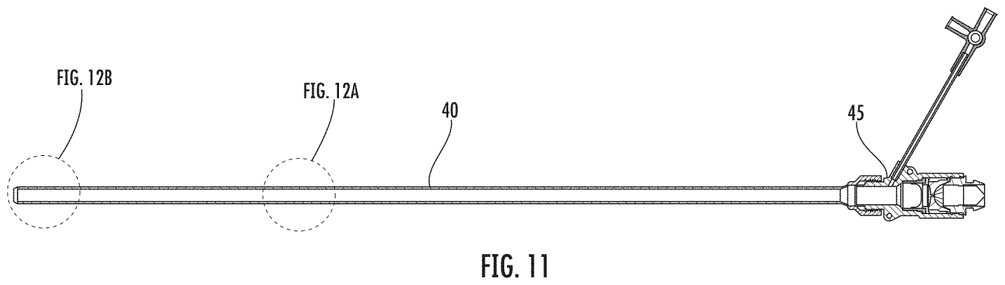

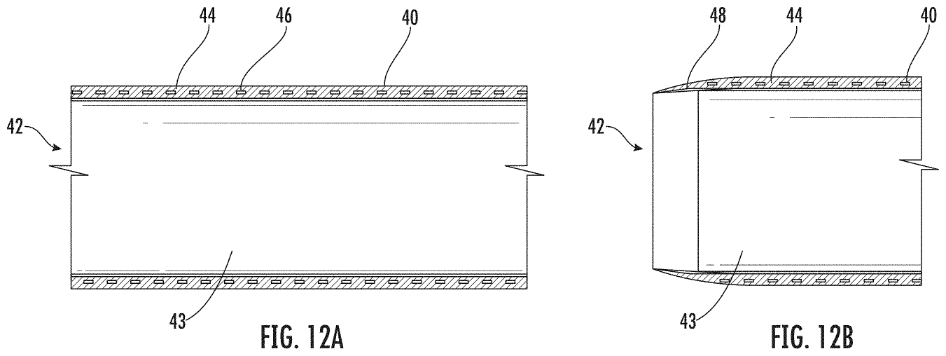

In some examples, the inner layer 40 includes a reinforcing member 46 such as a coil, braid, mesh or other structure for providing stiffness and flexibility to the inner layer 40. The reinforcing member 46 maintains the diameter of the central lumen 42 of the inner layer 40 when traversing bends in the patient's vasculature and prevents kinking. FIG. 11 provides a side cross-section view of the inner layer 40 without the introducer 26. FIGS. 12A and 12B are enlarged side cross-section views of the inner layer 40 without the introducer 26. As illustrated in FIGS. 11-12B, the inner layer 40 is defined by an elongated tubular member 44 with the reinforcing member 46 embedded therein. In other examples, the reinforcing member 46 is provided on the outer surface of the inner layer 40 or on the inner surface of the central lumen 42 of the inner layer 40. The reinforcing member 46 extends along the entire length of the inner layer 40, as provided in FIG. 11. In other examples, the reinforcing member 46 extends along a portion of the entire length of the inner layer 40.

As described above, the reinforcing member 46 can include a braid or mesh structure. The mesh structure comprises a woven mesh tube, a cut mesh tube, or a combination thereof. In some examples, the braid and/or mesh has a uniform braid density along the entire length of the reinforcing member. In other examples, the braid and/or mesh has a varying braid density along the entire length of the reinforcing member. The braid and/or mesh has a pick count ranging between about 4 picks per inch and about 50 picks per inch. In some examples, the pick count is consistent along an entire length of the inner layer 40. In other examples, the pick count varies along an entire length of the inner layer 40. The braid and/or mesh can define a diamond-shaped pattern, or any other pattern suitable for resisting axial compression applied to the inner layer 40, while facilitating bending of the inner layer 40 in a direction away from the longitudinal axis of the inner layer 40. The braid and/or mesh comprises at least one filament that is a round filament or a flat filament. When a round filament, the filament has a diameter ranging between 0.001 inches and about 0.015 inches. When a flat filament, the filament has a height ranging between 0.001 inches and 0.015 inches and a width ranging between about 0.001 inches and about 0.015 inches. The braid and/or mesh comprises at least one filament composed of a material comprising stainless steel, nitinol, a polymer material, or a composite material. Similarly, the braid and/or mesh comprises at least one filament composed of a material comprising a polymeric material including, for example, a polyolefin, polyamide fiber, or combinations thereof. The polymeric material can also comprise a polyester, a nylon, or a combination thereof.

As described above and illustrated in FIGS. 12A and 12B, the reinforcing member 46 can include a coil. Similar to the braid/mesh structure, the coil is wound to resist axial compression applied to the inner layer, while facilitating bending of the inner layer 40 in a direction away from the longitudinal axis of the inner layer 40. The coil includes a coil winding which provides compressive stiffness to the inner layer 40 during axial compressive loads. The coil has a plurality of tightly wound turns of the coil winding. As provided in FIG. 12A, the coil winding has a constant pitch along the axial length of the coil. In other examples, the coil winding has a varying pitch along an entire length of the coil winding/coil. For example, the pitch of the coil winding a distal portion of the coil (adjacent the distal end of the inner layer 40) can be less than a pitch of the coil winding at the proximal portion of the coil (adjacent the proximal end of the inner layer 40). The coil winding has a pitch angle ranging between about 10 degrees and about 180 degrees. As illustrated in FIGS. 12A-12B, a gap/spacing is provided between adjacent turns of the coil winding. In other examples, the coil is tightly wound such that the adjacent turns of the coil winding contact, i.e., the pitch is zero/functionally zero and the outer surface of the adjacent turns of the coiled winding contact along a circumferential length of the coiled winding. The pitch of the coil winding ranges between 1 turns per inch and 40 turns per inch. In some examples, the pitch ranges between 30 turns per inch and 40 turns per inch. The coil winding has a diameter ranging between 0.001 inches and about 0.015 inches. Similar to the braid/mesh structure, the coil winding is composed of a material comprising stainless steel, nitinol, a polymer material, or a composite material.

While the tubular member 44/inner layer 40 defines a generally constant cylindrically-shaped structure, the distal end of the inner layer 40 can include an atraumatic, non-expandable distal tip 48. As illustrated in FIGS. 11 and 12B, the distal tip 48 defines a tubular structure with a slightly tapering or frusto-conical distal end. The outer surface of the distal tip 48 includes a distally tapering and/or curved shape such that a diameter of the distal tip 48 adjacent the tubular member 44 is greater than the diameter of the distal end of the distal tip 48. In some examples, the inner surface of the central lumen 42 of the distal tip 48 includes a distally tapering and/or curved shape. In other examples, the central lumen 42 of the distal tip 48 has a constant inner diameter. As illustrated in FIG. 12B, the thickness of the distal tip 48 decreases between the proximal and distal ends.

The distal tip 48 is formed of a non-elastic material. Because the distal tip 48 does not expand, this prevents it from folding during retrieval of the delivery apparatus 10/prosthetic heart valve 12. In some examples, the distal tip 48 is formed from the same material as the tubular member 44. In other examples, the distal tip 48 is formed from a different elastic material as the tubular member 44. The distal tip 48 is integrally formed with the tubular member 44. For example, the distal tip 48 can be co-extruded with the tubular member 44 or reflowed from the existing tubular member 44 into the desired shape. In other examples, the distal tip 48 is separately formed and coupled to the tubular member 44. For example, the distal tip 48 can be extruded/molded separately and coupled to the tubular member 44 using heat processing. Regardless of whether integrally or separately formed with the tubular member 44, the distal tip 48 has a lower hardness/is softer than a proximal end portion of the inner layer 40.

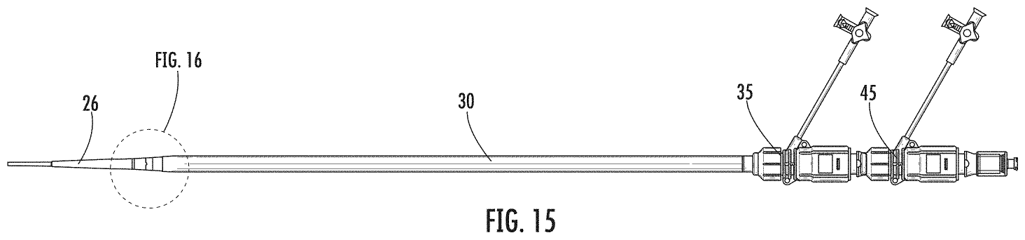

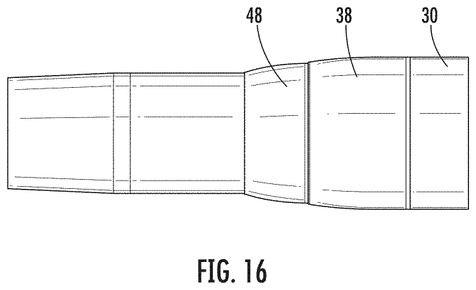

The structure of the distal tip 48 increases the structural rigidity of the distal end of the sheath assembly 22 and helps prevent buckling and/or folding of either the inner layer 40 and/or outer layer 30 during retrieval of the delivery apparatus 10/prosthetic heart valve 12. As illustrated in FIGS. 15 and 16, this structure also provides a smooth, tapered profile between the inner layer 40 and the introducer 26 for pushing the inner layer 40 and introducer 26 through the central lumen 32 of the outer layer 30.

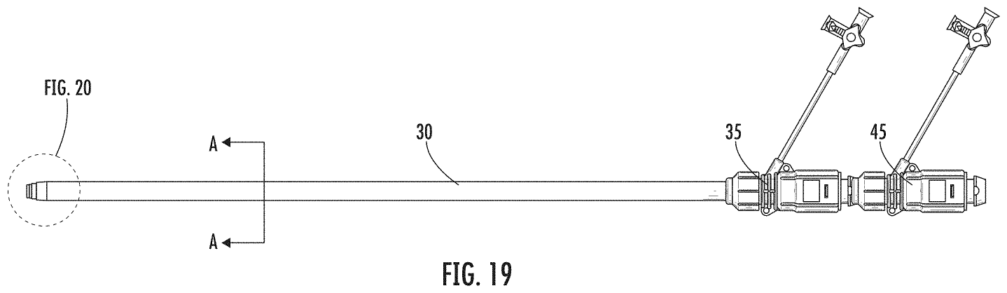

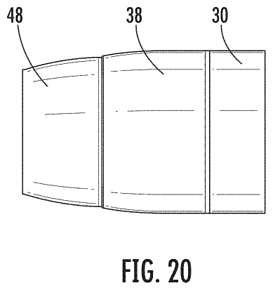

As will be described in more detail below and as illustrated in FIGS. 19-20, when the inner layer 40 is fully received within the central lumen 32 of the outer layer 30, a length of the inner layer 40 extends through and/or beyond a distal opening of the central lumen 32 of the outer layer 30. The protruding length of the inner layer 40 includes the distal tip 48. The protruding length can also include a portion of the tubular member 44 of the inner layer 40 adjacent the distal tip 48. Accordingly, the structure of the distal tip 48 blocks blood flow between the layers and provides a smooth, tapered profile for pushing the combined outer layer 30 and inner layer 40 through within the patient's blood vessel.

The inner and outer layers 30, 40 include hydrophilic coatings and/or lubricious liners to facilitate insertion into the patient's anatomy and movement between the inner and outer layers 30, 40, and reducing potential damage to the sheath and patient trauma. In some embodiments, the sheath assembly 22 can include an exterior hydrophilic coating on the outer surface of the outer layer 30 to facilitate insertion into a patient's vessel. Similarly, a hydrophilic coating can be provided on the outer surface of the inner layer 40 and/or the central lumen 32 of the outer layer 30 to reduce friction between the outer layer 30 and the inner layer 40 when the inner layer 40 is received and/or moved within the central lumen 32 of the outer layer 30. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N. V, Heerlen, the Netherlands), as well as other hydrophilic coatings (e.g., PTFE, polyethylene, polyvinylidine fluoride), are also suitable for use with the sheath assembly 22. Such hydrophilic coatings may also be included on the inner surface of the inner layer 40 to reduce friction between the sheath and the delivery apparatus 10/prosthetic heart valve 12, thereby facilitating use and improving safety. In some embodiments, a hydrophobic coating, such as Perylene, may be used on the outer surface of the outer layer 30 or the inner surface of the inner layer 40 in order to reduce friction.

As illustrated in FIGS. 12A-12B, some embodiments of a sheath assembly 22 include a lubricious liner 43 on the inner surface of the inner layer 40 to reduce friction between the inner layer 40 and a passing medical device. Examples of suitable lubricious liners include materials that can further reduce the coefficient of friction of the inner layer 40, such as PTFE, polyethylene, polyvinylidine fluoride, and combinations thereof. Suitable materials for a lubricious liner also include other materials desirably having a coefficient of friction of 0.5 or less, preferably 0.1 or less.

As discussed above, the outer layer 30 and the inner layer 40 are each coupled at their proximal ends to a hub 35, 45. The hub(s) can function as a handle for the expandable sheath assembly 22. Examples of such hubs is described in U.S. Provisional Patent Application No. 63/077,899 (titled “Reverse Bayonet Locking Hub,” filed Sep. 14, 2020), the disclosure of which is hereby incorporated by reference. In certain embodiments, the outer layer hub 35 can include a hemostasis valve that forms a seal around the outer surface of the guide catheter 14 once inserted through the housing to prevent leakage of pressurized blood. Similarly, the inner layer hub 45 can include a hemostasis valve that forms a seal around the outer surface of the outer layer 30 to prevent leakage of pressurized blood therebetween.

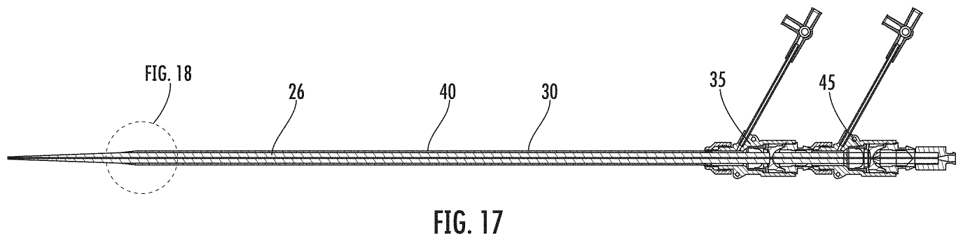

When the inner layer 40 is fully inserted within the central lumen 32 of the outer layer 30 (FIGS. 15, 17, 19) the inner and outer layers 30, 40 are coupled at their proximal ends. For example, hub 35 and hub 45 can include a locking or engagement feature for removably coupling when the inner layer 30 is fully inserted within the outer layer. When in a locked position, the locking feature fixes the axial and rotational position of the inner layer 40 with respect to the outer layer 30.

In some example sheath assemblies 22, the outer and/or inner layers 30, 40 can include a radiopaque marker which can be provided to improve visibility under fluoroscopy or other similar techniques. The radiopaque marker can be located proximate the distal tip 38, 48 of the outer and/or inner layers 30, 40. Additionally or alternatively, the radiopaque marker can be provided along a length of the tubular member 34, 44 of the outer and/or inner layers 30, 40. The radiopaque marker can be in the form of a disk or tag embedded within the outer and/or inner layers 30, 40. The radiopaque marker can be a radiopaque ring provided circumferentially around the outer and/or inner layers 30, 40. The radiopaque marker can be incorporated into one or more of the axial supports 36 of the outer layer 30 and/or the reinforcing member 46 of the inner layer 40.

A method of delivering a medical device using a two-component sheath assembly 22 as illustrated in FIG. 2 is disclosed herein. The method comprises inserting an outer layer 30 of the sheath assembly 22 at least partially into the vasculature of the patient and advancing the outer layer 30 to the implantation site within the blood vessel. The outer layer 30 includes an elongated tubular member 34 with integrated axial supports 36 extending longitudinally therein. An introducer 24 is provided within the central lumen 32 of the outer layer 30 to provide column strength during insertion and positioning. The outer layer 30 is an expandable sheath movable between a non-expanded configuration and an expanded configuration. During insertion and positioning, the outer layer 30 is in the non-expanded configuration. The relative low profile and softness of the outer layer 30 enables it to easily bend along the vascular path with minimal risk of trauma.

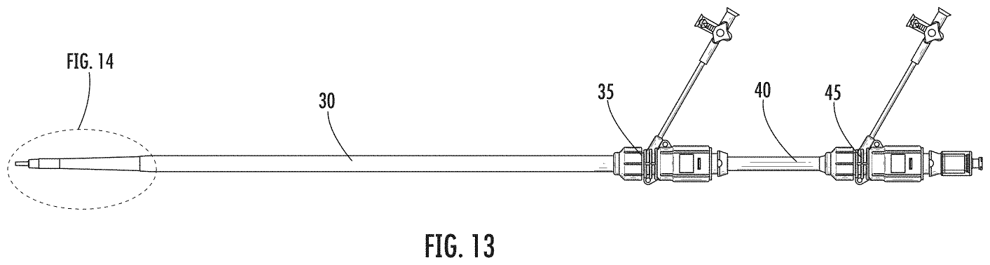

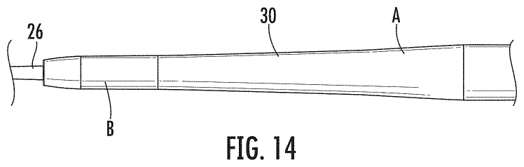

Once positioned at the treatment site, the introducer 24 is removed from the outer layer 30 and the inner layer 40 is advanced within the central lumen 32 of the outer layer 40. An introducer 26 is provided within the central lumen 42 of the inner layer 40. FIG. 13 illustrates a side view of the inner layer 40 partially inserted into the outer layer 30. During insertion, at least a portion of the outer layer 30 is expanding from the non-expanded configuration towards the expanded configuration by the radially outward force exerted on an inner surface of the outer layer 30 by the advancement of the inner layer 40. FIG. 14 is an enlarged partial cross-section view of the distal end of the sheath assembly 22. As illustrated in FIG. 14, the expanded portion (A) of the outer layer 30 corresponds to the axial location of the inner layer 40 within the central lumen 32. The non-expanded portion (B) of the outer layer 30 corresponds to the portion of the central lumen 32 where the inner layer is not present. Because the outer layer 30 is biased to the non-expanded configuration, the non-expanded portion (B) of the outer layer 30 has a smaller diameter than the expanded portion (A).

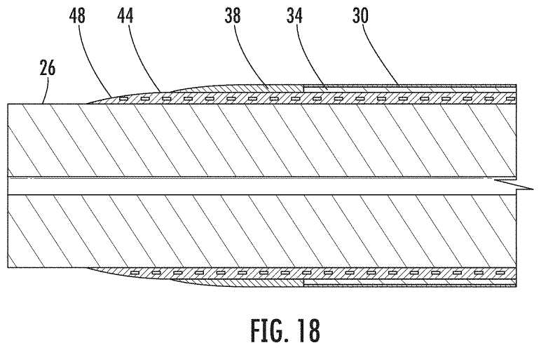

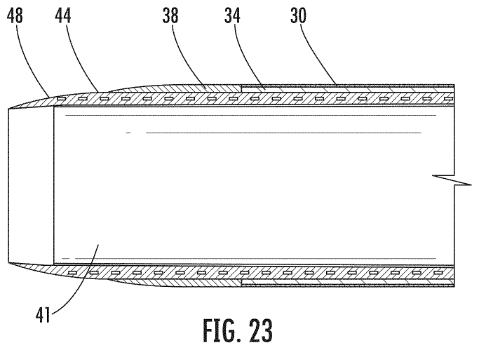

The inner layer 40 is advanced fully into the outer layer 30 such that a portion of the outer layer 30 extends through and/or beyond the distal opening of the outer layer 30. FIGS. 15, 17, and 19 provide side and cross-section views of the inner layer 40 fully inserted into the outer layer 30. As provided in the enlarged side and cross-section views of FIGS. 16, 18, 20 and 23 the distal tip 48 (and a portion of the tubular member 44) of the inner layer 40 extends beyond the distal opening of the outer layer 30. The structure of the distal tip 48 provides a smooth transition between the introducer 26 and the inner layer 40. The structure of the distal tip 38 provides a smooth transition between the inner layer 40 and the outer layer 30. This structure of the distal tips 38, 48 blocks blood flow between the layers and provides a smooth, tapered profile for pushing/positioning the combined outer layer 30 and inner layer 40 through within the patient's blood vessel.

The insertion of the inner layer 40 into the outer layer 30 gently expands the outer layer 30 to create and enlarge a central lumen for the delivery apparatus 10. As described above, the outer layer 30 includes axial supports 36 spaced circumferentially around the elongated tubular member 34. During the (local) expansion the outer layer 30 to/towards the expanded configuration, the circumferential spacing between the axial supports 36 increases. FIG. 21 provides a cross section view of the inner and outer layers 40, 30 of FIG. 19 along section line A-A. As illustrated in FIG. 21, a portion of the outer surface of the axial supports 36 protrude from an inner surface of the central lumen 32 of the outer layer 30. The outer surface of the axial supports 36 provide a bearing surface between the outer layer 30 and the inner layer 40, reducing friction and push force when the inner layer 40 is advanced within and/or removed from the central lumen 32 of the outer layer 30.

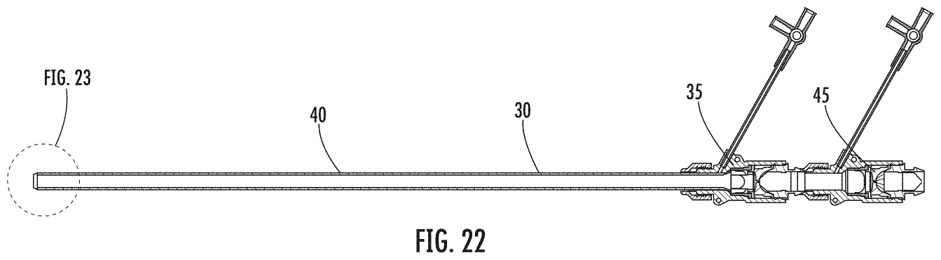

With the inner layer 40 fully inserted into the outer layer 30, the introducer 26 can be removed from the central lumen 42 of the inner layer 40. FIGS. 19 and 22 provide side and cross-section views of the inner layer 40 fully inserted into the outer layer 30 with the introducer 26 removed.

The inner layer 40 can be coupled to the outer layer 30 such that the axial and rotational positions of each are fixably coupled with respect to the other. For example, the proximal end of the inner layer 40 is coupled with the proximal end of the outer layer 30 at the outer layer hub 35 and inner layer hub 45.

The delivery apparatus 10 and prosthetic heart valve 12 are advanced to the treatment site via the central lumen 42 of the inner layer 40. The prosthetic heart valve 12 can include a stent mounted heart valve mounted in a radially crimped state on a delivery apparatus 10. The delivery apparatus 10 and the prosthetic heart valve 12 are advanced through the central lumen 42 of the inner layer 40 and into the vasculature. The prosthetic heart valve 12 is then implanted at the treatment site within the patient. If a stent mounted heart valve is used, the heart valve is expanded after it exits the central lumen 42 of the inner layer 40.

With the prosthetic heart valve 12 implanted, the delivery apparatus 10 is removed from the central lumen 42 of the inner layer 40.

The inner layer 40 is then removed from the central lumen 32 of the outer layer 30. Because the outer layer 30 is biased to the non-expanded configuration, as the inner layer 40 is removed, the outer layer 30 locally contracts from the expanded configuration at least partially back to/towards the non-expanded configuration. As the inner layer 40 is removed, and the outer layer 30 contracts, the circumferential spacing between the axial supports 36 in the outer layer 30 decreases to/toward the original, non-expanded spacing.

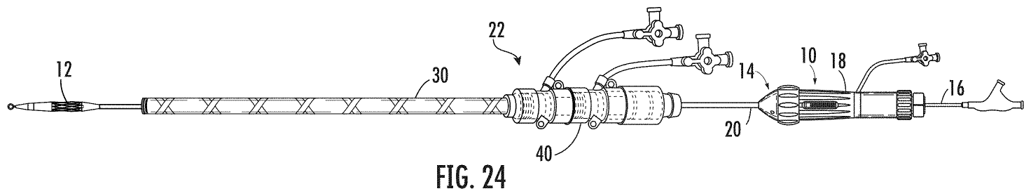

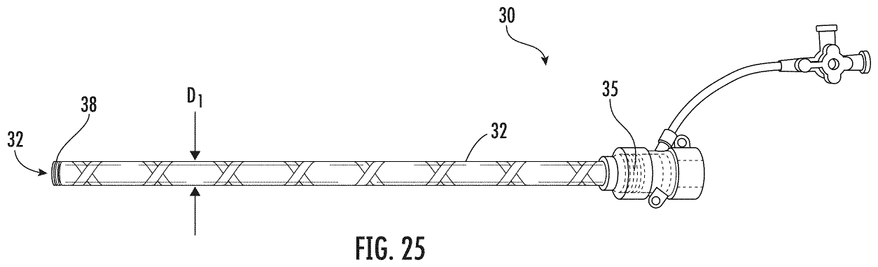

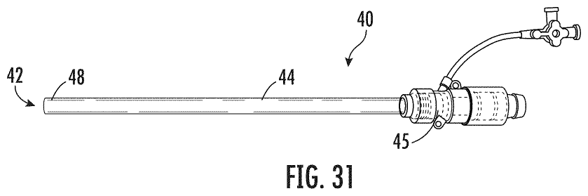

FIG. 24 illustrates a second example expandable sheath assembly 22 (which can be referred to as an introducer sheath device or assembly) that can be used to introduce the delivery apparatus 10 and the prosthetic device 12 into a patient's body. With respect to the expandable sheath assembly of FIG. 2, like element numbers will be used to describe like features. The expandable sheath assembly 22 includes an outer tubular layer 30 (also referred to herein as outer layer, sheath outer layer and expandable outer layer) and an inner tubular layer 40 (also referred to herein as inner layer, reinforced layer) received within a central lumen 32 (FIG. 25) of the outer layer 30 and movable therein. The expandable sheath assembly 22, and in particular the inner layer 40, has a central lumen 42 (FIG. 31) to guide passage of the delivery apparatus 10.

During use a distal end of the outer layer 30 is passed through the skin of the patient and is inserted into a vessel, such as the femoral artery. As will be described in more detail below, the outer layer 30 provides a small diameter/profile sheath that can be inserted into the patient's vasculature with minimal risk of trauma. The inner layer 40, having a larger diameter than outer layer 30, is inserted into the central lumen 32 of the outer layer 30 temporarily expanding the outer layer 30 from the non-expanded configuration to an expanded configuration, and providing an enlarged central lumen 42 to allow passage of the delivery apparatus 10 and the prosthetic heart valve 12. The enlarged diameter of the central lumen 42 of the inner layer 40 decreases the required push force to move the prosthetic heart valve 12 through the sheath assembly 22.

The delivery apparatus 10, with the prosthetic heart valve 12 mounted thereon, are then inserted through the hub(s) and the inner layer 40, and advanced through the patient's vasculature to the treatment site, where the implant is to be delivered and implanted within the patient

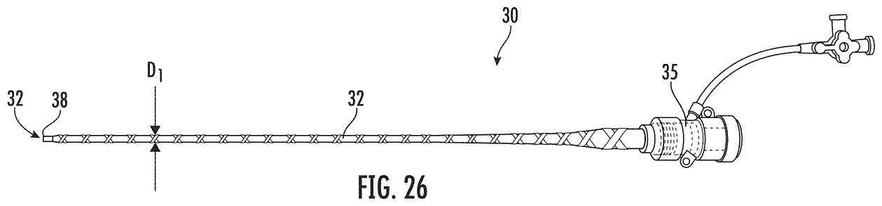

FIGS. 25 and 26 provide side views of the outer layer 30 of FIG. 24. As described above, the outer layer 30 provides a relatively flexible layer with a small diameter/profile in non-expanded configuration. During insertion, an introducer is provided within the central lumen 32 of the outer layer 30 to maintain the shape of the sheath and provide the necessary column strength during insertion, helping prevent the outer layer 30 from kinking and/or collapsing. The outer layer 30 protects vasculature, prevents calcification dislodgement, and transfers the potentially traumatic axial force of insertion to safe, radial force on the vasculature.

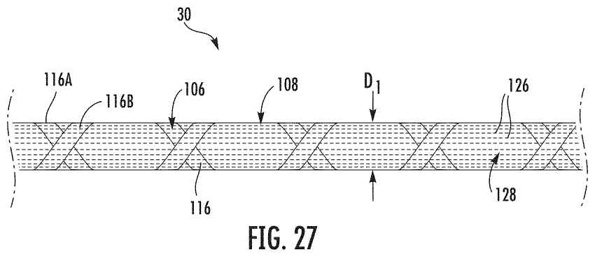

In certain embodiments, the outer layer 30 can comprise a plurality of coaxial layers extending along at least a portion of the length of the outer layer 30. Example expandable sheaths are described, for example, in U.S. patent application Ser. No. 16/378,417, entitled “Expandable Sheath,” U.S. Provisional Patent Application No. 62/912,569, entitled “Expandable Sheath,” and U.S. Provisional Patent Application No. 63/091,722, entitled “Radiopaque Foil Encapsulated within an expandable Sheath” the disclosures of which are herein incorporated by reference. The structure of the coaxial layers is described in more detail below with respect to FIGS. 27-30.

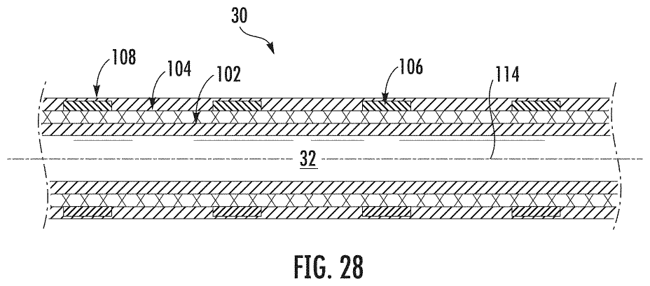

FIG. 27 illustrates the expandable sheath outer layer 30 in greater detail. With reference to FIG. 27, the outer layer can have a natural, unexpanded outer diameter D₁. In certain embodiments, the sheath outer layer 30 can comprise a plurality of coaxial layers extending along at least a portion of the length of the outer layer 30. For example, with reference to FIG. 28, the sheath outer layer 30 can include a first layer 102 (also referred to as first polymeric layer) and a second layer 104 disposed around and radially outward of the first layer 102. As will be described in more detail below, the second layer 104 can be a braided layer comprising a plurality of filaments braided together. In some examples, a third layer 106 is disposed around and radially outward of the second layer 104, and a fourth layer 108 disposed around and radially outward of the third layer 106. In the illustrated configuration, the inner polymeric layer 102 can define the central lumen 32 of the outer layer 30 extending along a central axis 114.

Referring to FIG. 27, when the sheath outer layer 30 is in an unexpanded state, the inner polymeric layer 102 and/or the outer layer 108 can form longitudinally extending folds or creases such that the surface of the sheath comprises a plurality of ridges 126 (also referred to herein as “longitudinally extending folds”). The ridges 126 can be circumferentially spaced apart from each other by longitudinally extending valleys 128. When the sheath expands beyond its natural diameter D₁ (e.g., when the inner layer 40 is passed through the central lumen 32 of the outer layer 30), the ridges 126 and the valleys 128 can level out or be taken up as the surface radially expands and the circumference increases, as further described below. When the sheath outer layer 30 collapses back to its natural/unexpanded diameter, the ridges 126 and valleys 128 can reform.

In certain embodiments, the inner polymeric layer 102 and/or the outer layer 108 can comprise a relatively thin layer of polymeric material. For example, in some embodiments the thickness of the inner polymeric layer 102 can be from 0.01 mm to 0.5 mm, 0.02 mm to 0.4 mm, or 0.03 mm to 0.25 mm. In certain embodiments, the thickness of the outer layer 108 can be from 0.01 mm to 0.5 mm, 0.02 mm to 0.4 mm, or 0.03 mm to 0.25 mm.

In certain examples, the inner polymeric layer 102 and/or the outer layer 108 can comprise a lubricious, low-friction, and/or relatively non-elastic material. In particular embodiments, the inner polymeric layer 102 and/or the outer layer 108 can comprise a polymeric material having a modulus of elasticity of 400 MPa or greater. Exemplary materials can include ultra-high-molecular-weight polyethylene (UHMWPE) (e.g., Dyneema®), high-molecular-weight polyethylene (HMWPE), or polyether ether ketone (PEEK). With regard to the inner polymeric layer 102 in particular, such low coefficient of friction materials can facilitate passage of the prosthetic device through the central lumen 32. Other suitable materials for the inner and outer layers can include polytetrafluoroethylene (PTFE), expanded polytetrafluoroethylene (ePTFE), ethylene tetrafluoroethylene (ETFE), nylon, polyethylene, polyether block amide (e.g., Pebax), and/or combinations of any of the above. Some embodiments of the outer layer 30 can include a lubricious liner on the inner surface of the inner polymeric layer 102. Examples of suitable lubricious liners include materials that can further reduce the coefficient of friction of the inner polymeric layer 102, such as PTFE, polyethylene, polyvinylidine fluoride, and combinations thereof. Suitable materials for a lubricious liner also include other materials desirably having a coefficient of friction of 0.1 or less.

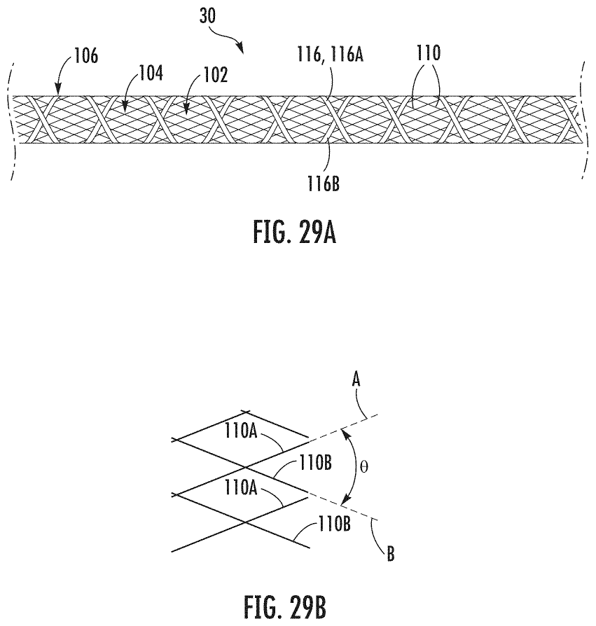

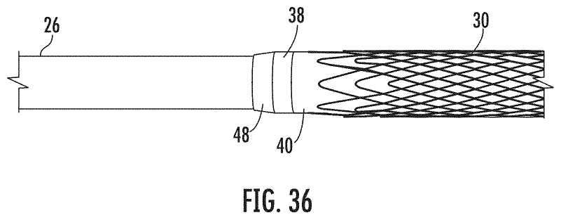

In certain embodiments, the second layer 104 can be a braided layer. FIGS. 29A and 29B illustrate the sheath outer layer 30 with the outer layer 108 removed to expose the elastic third layer 106. With reference to FIGS. 29A, 29B and 36, the braided second layer 104 can comprise a plurality of members or filaments 110 (e.g., metallic or synthetic wires or fibers) braided together. The braided second layer 104 can have any desired number of filaments 110, which can be oriented and braided together along any suitable number of axes. For example, with reference to FIG. 29B, the filaments 110 can include a first set of filaments 110A oriented parallel to a first axis A, and a second set of filaments 110B oriented parallel to a second axis B. The filaments 110A and 110B can be braided together in a biaxial braid such that filaments 110A oriented along axis A form an angle θ with the filaments 110B oriented along axis B. In certain embodiments, the angle θ can be from 5° to 70°, 10° to 60°, 10° to 50°, or 10° to 45°. In the illustrated embodiment, the angle θ is 45°. In other embodiments, the filaments 110 can also be oriented along three axes and braided in a triaxial braid or oriented along any number of axes and braided in any suitable braid pattern.

The braided second layer 104 can extend along substantially the entire length L of the sheath outer layer 30, or alternatively, can extend only along a portion of the length of the sheath outer layer 30. In particular embodiments, the filaments 110 can be wires made from a superelastic metal (e.g., Nitinol, stainless steel, etc.), or any of various polymers or polymer composite materials, such as carbon fiber. In certain embodiments, the filaments 110 can be round, and can have a diameter of from 0.01 mm to 0.5 mm, 0.03 mm to 0.4 mm, or 0.05 mm to 0.25 mm. In other embodiments, the filaments 110 can have a flat cross-section with dimensions of 0.01 mm×0.01 mm to 0.5 mm×0.5 mm, or 0.05 mm×0.05 mm to 0.25 mm×0.25 mm. In one embodiment, filaments 110 having a flat cross-section can have dimensions of 0.1 mm×0.2 mm. However, other geometries and sizes are also suitable for certain embodiments. If braided wire is used, the braid density can be varied. Some embodiments have a braid density of from ten picks per inch to eighty picks per inch, and can include eight wires, sixteen wires, or up to fifty-two wires in various braid patterns. In other embodiments, second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an elastic material layer). In certain embodiments, the elastic third layer 106 can be configured to apply force to the underlying first layer 102 and second layer 104 in a radial direction (e.g., toward the central axis 114 of the sheath outer layer 30) when the sheath outer layer 30 expands beyond its natural diameter by passage of the delivery apparatus 10 and/or inner layer 40 through the outer layer 30. Stated differently, the elastic third layer 106 can be configured to apply encircling pressure to the layers of the sheath outer layer 30 beneath the elastic third layer 106 to counteract expansion of the sheath outer layer 30. The radially inwardly directed force is sufficient to cause the sheath outer layer 30 to collapse radially back to its unexpanded state after the delivery apparatus 10 and/or inner layer 40 is passed through the sheath outer layer 30. It is understood, however, that the elastic third layer 106 can be optional. And also described herein are the embodiments where this third elastic layer is not present, while all other layers described herein are. It is also understood that this description includes all various combinations of the layers, and unless it is stated otherwise, some of the described herein layers (liners) can be present while others can be absent.

In the illustrated embodiment, the elastic third layer 106 can comprise one or more members configured as strands, ribbons, or bands 116 helically wrapped around the braided second layer 104. For example, in the illustrated embodiment the elastic third layer 106 comprises two elastic bands 116A and 1166 wrapped around the braided layer with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some embodiments, the elastic layer can comprise an elastomeric material having a modulus of elasticity of 200 MPa or less. In some embodiments, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc. In lieu of, or in addition to, the elastic third layer 106, the sheath outer layer 30 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In other embodiments, the elastic third layer 106 can also be radially outward of the polymeric outer layer 108.

In certain embodiments, one or both of the inner polymeric layer 102 and/or the outer layer 108 can be configured to resist axial elongation of the sheath outer layer 30 when the sheath outer layer 30 expands. More particularly, one or both of the inner polymeric layer 102 and/or the outer layer 108 can resist stretching against longitudinal forces caused by friction between a prosthetic device and the inner surface of the central lumen 32 of the sheath outer layer 30 such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 29B, the filaments 110A and 110B of the braided layer can be allowed to move angularly relative to each other such that the angle θ changes as the sheath expands and contracts. This, in combination with the longitudinally extending ridges 126 (folds) in the polymeric inner layer 102 and outer layer 108, can allow the central lumen 32 of the sheath outer layer 30 to expand as a delivery apparatus 10, inner layer 30, or a prosthetic heart valve 12 is advanced through it.

For example, in some embodiments the inner polymeric layer 102 and the outer layer 108 can be heat-bonded during the manufacturing process such that the braided second layer 104 and the elastic third layer 106 are encapsulated between the polymeric inner layer 102 and outer layer 108. More specifically, in certain embodiments the inner polymeric layer 102 and the outer polymeric outer layer 108 can be adhered to each other through the spaces between the filaments 110 of the braided second layer 104 and/or the spaces between the elastic bands 116. The polymeric layers 102 and 108 can also be bonded or adhered together at the proximal and/or distal ends of the sheath. In certain embodiments, the polymeric layers 102 and 108 are not adhered to the filaments 110. This can allow the filaments 110 to move angularly relative to each other, and relative to the layers 102 and 108, allowing the diameter of the braided second layer 104, and thereby the diameter of the sheath outer layer 30, to increase or decrease. As the angle θ between the filaments 110A and 110B changes, the length of the braided second layer 104 can also change. For example, as the angle θ increases, the braided second layer 104 can foreshorten, and as the angle θ decreases, the braided second layer 104 can lengthen to the extent permitted by the areas where the polymeric layers 102 and 108 are bonded. However, because the braided second layer 104 is not adhered to the polymeric layers 102 and 108, the change in length of the braided second layer 104 that accompanies a change in the angle θ between the filaments 110A and 110B does not result in a significant change in the length L of the sheath.

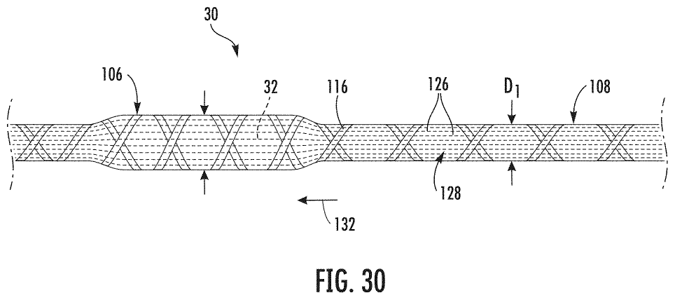

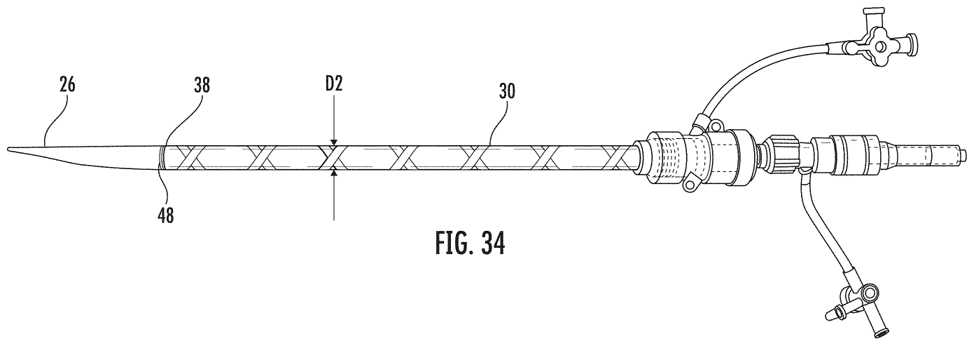

FIG. 30 illustrates a local radial expansion of the sheath outer layer 30 as a prosthetic heart valve 12 is passed through the sheath outer layer 30 in the direction of arrow 132 (e.g., distally). FIG. 34 illustrates the local radial expansion of the sheath outer layer 30 when the inner layer 40 and introducer 26 are received within the central lumen 32 of the outer layer 30. As the prosthetic heart valve 12 and/or inner layer 40 is advanced through the sheath outer layer 30, the outer layer 30 can resiliently expand to a second diameter D₂ that corresponds to a size or diameter of the prosthetic device/inner layer 40. In some examples, the diameter (D₁) of the central lumen 32 of the sheath outer layer 30 in the non-expanded configuration ranges between about 8 F and about 14 F, and the inner diameter (D₂) of the central lumen 32 in the expanded configuration ranges from about 20 F to about 28 F. In some examples, the inner diameter of the central lumen 32 of the sheath outer layer 30 in the expanded configuration ranges from about 14 Fr to about 24 Fr.

As the prosthetic device 12/inner layer 40 is advanced through the sheath outer layer 30, the prosthetic device 12/inner layer 40 can apply longitudinal force to the sheath in the direction of motion by virtue of the frictional contact between the prosthetic device 12/inner layer 40 and the inner surface of the sheath outer layer 30. However, as noted above, the inner polymeric layer 102 and/or the outer polymeric outer layer 108 can resist axial elongation such that the length L of the sheath outer layer 30 remains constant, or substantially constant. This can reduce or prevent the braided second layer 104 from lengthening, and thereby constricting the central lumen 32.

Meanwhile, the angle θ between the filaments 110A and 110B can increase as the sheath outer layer 30 expands to the second diameter D₂ to accommodate the prosthetic valve/inner layer 40. This can cause the braided second layer 104 to foreshorten. However, because the filaments 110 are not engaged or adhered to the polymeric layers 102 or 108, the shortening of the braided second layer 104 attendant to an increase in the angle θ does not affect the overall length L of the sheath outer layer 30. Moreover, because of the longitudinally extending ridges 126 (folds) formed in the polymeric layers 102 and 108, the layers 102 and 108 can expand to the second diameter D₂ without rupturing, in spite of being relatively thin and relatively non-elastic. In this manner, the sheath outer layer 30 can resiliently expand from its natural diameter D₁ to a second diameter D₂ that is larger than the diameter D₁ as a prosthetic device and/or inner layer 40 is advanced through the sheath outer layer 30, without lengthening, and without constricting. Thus, the force required to push the prosthetic implant through the sheath outer layer 30 is significantly reduced.

Additionally, because of the radial force applied by the elastic third layer 106, the radial expansion of the sheath outer layer 30 can be localized to the specific portion of the sheath outer layer 30 occupied by the prosthetic device/inner layer 40. For example, with reference to FIGS. 30 and 34, as the prosthetic device 12/inner layer 40 moves distally through the sheath outer layer 30, the portion of the sheath outer layer 30 immediately proximal to the prosthetic device 12/inner layer 40 can radially collapse back to the initial diameter D₁ under the influence of the elastic third layer 106. The polymeric layers 102 and 108 can also buckle as the circumference of the sheath outer layer 30 is reduced, causing the ridges 126 and the valleys 128 to reform. This can reduce the size of the sheath outer layer 30 required to introduce a prosthetic device of a given size. Additionally, the temporary, localized nature of the expansion can reduce trauma to the blood vessel into which the sheath outer layer 30 is inserted, along with the surrounding tissue, because only the portion of the sheath outer layer 30 occupied by the prosthetic device and/or inner layer 40 expands beyond the sheath's natural diameter and the sheath outer layer 30 collapses back to the initial diameter once the device has passed. This limits the amount of tissue that must be stretched in order to introduce the prosthetic device and/or inner layer 40, and the amount of time for which a given portion of the vessel must be dilated.

In alternative embodiments, the sheath outer layer 30 may optionally include the polymeric inner layer 102 without the polymeric outer layer 108, or the polymeric outer layer 108 without the polymeric inner layer 102, depending upon the particular characteristics desired.

In some examples, the inner layer 40 includes a tear away feature for facilitate removing the inner layer 40 from the outer layer 30. With the larger diameter inner layer 40 removed, the expandable outer layer 30 can return to/towards the non-expanded, reduced diameter, configuration. The medical procedure will then continue with the reduced diameter outer layer 30 in place within the patient's vasculature. Example tear away features include one or more longitudinally extending slits, weakened portions, scorelines, pull wires, or other features to cause the inner layer 40 to break or split so that it can be separated from the outer layer 30. In general, the tear away feature extends from the proximal end of the inner layer 40 toward the distal end. The tear away feature can extend along the entire length of the inner layer 40 or along a portion thereof.

While the outer surface of the tubular member 34/outer layer 30 includes has a generally constant cylindrical outer diameter, the distal end of the outer layer 30 can include an atraumatic, expandable distal tip 38. As illustrated in FIGS. 25, 26 and 36, the distal tip 38 defines a tubular structure with a slightly tapering or frusto-conical distal end. The outer surface of the distal tip 38 includes a distally tapering and/or curved shape such that a diameter of the distal tip 38 adjacent the tubular member 34 is greater than the diameter of the distal end of the distal tip 38. In some examples, the inner surface of the central lumen of the distal tip 38 includes a distally tapering and/or curved shape. In other examples, the central lumen of the distal tip 38 has a constant inner diameter. In some examples, the thickness of the distal tip 38 decreases between the proximal and distal ends. The distal tip 38 is formed of an elastomeric material that expands with the tubular member 34 between the non-expanded and expanded configurations. In some examples, the distal tip 38 is formed from the same material as the tubular member. In other examples, the distal tip 38 is formed from a different elastic material as the tubular member. The distal tip 38 is integrally formed with the tubular member 34. For example, the distal tip 38 can be co-extruded with the tubular member 34 or reflowed from the existing tubular member 34 into the desired shape. In other examples, the distal tip 38 is separately formed and coupled to the tubular member 34. For example, the distal tip 38 can be extruded/molded separately and coupled to the tubular member 34 using heat processing.

The structure of the distal tip 38 of the outer layer 30 helps to increase the structural rigidity of the distal end of the tubular wall structure, blocks blood flow between the layers, and provides a smooth, tapered profile for pushing through tissue when advanced over a wire or introducer.

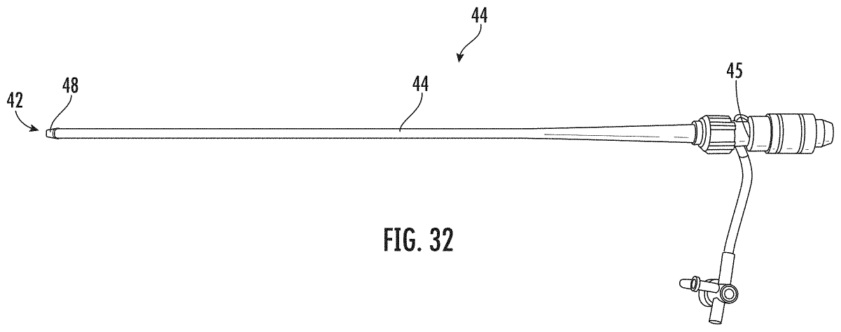

As described above, the expandable sheath assembly 22 includes an inner layer 40. FIGS. 31 and 32 provide side views of the inner layer 40 of FIG. 25. As described above, when the inner layer 40 is inserted into the central lumen 32 of the outer layer 30, it causes the outer layer 30 to locally expand from the non-expanded configuration to the expanded configuration by the radially outward force exerted on the surface of the central lumen 32 of the outer layer 30 by the inner layer 40. The enlarged diameter of the central lumen 42 of the inner layer 40 is large enough that the delivery apparatus 10/prosthetic heart valve 12 can pass through without additional expansion of the outer layer 30 and/or the inner layer 40, decreasing the required push force to move the device through the sheath assembly 22. As the inner layer 40 is removed from the outer layer 30, the outer layer 30 locally contracts from the expanded configuration at least partially back to the non-expanded configuration.

In general, the inner layer 40 does not expand radially or experiences minimal radial expansion. In some examples, the radial expansion of the inner layer 40 is less than 1%. The inner diameter of the central lumen 42 of the inner layer 40 ranges between about 16 F and about 28 F. In some examples, the inner diameter ranges between about 20 F and about 28 F. In use, the clinician can select an inner layer 40 having a diameter based on the particular therapy and size of the delivery apparatus 10. Generally, the diameter is selected as one greater than the outer diameter of the delivery apparatus 10 to minimize trauma and unnecessary expansion, but large enough that the delivery apparatus 10 and prosthetic heart valve 12 can pass freely therethrough.

In certain examples, the inner layer 40 can comprise a lubricious, low-friction, and/or relatively non-elastic material. The inner layer 40 provides stiffness and flexibility to the outer layer 30 when the sheaths are assembled and maintains the diameter of the central lumen 42 of the inner layer 40 when traversing bends in the patient's vasculature. In general, the inner layer 40 has a higher column strength than the outer layer 30. The inner layer 40 also has a lower rigidity and is more flexible than the outer layer 30.

Exemplary materials can include ultra-high-molecular-weight polyethylene (UHMWPE) (e.g., Dyneema®), high-molecular-weight polyethylene (HMWPE), high density polyethylene (HDPE), or polyether ether ketone (PEEK). With regard to the inner layer 40 in particular, the use of low coefficient of friction materials can facilitate passage of the prosthetic device through the central lumen 42. Other suitable materials for the inner layer 40 can include polytetrafluoroethylene (PTFE), polyethylene terephthalate (PET), expanded polytetrafluoroethylene (ePTFE), ethylene tetrafluoroethylene (ETFE), nylon, polyethylene, polyether block amide (e.g., Pebax), nylon, fluorinated ethylene propylene (FEP), polypropylene (PP), and/or combinations of any of the above.

While the tubular member 44/inner layer 40 defines a generally constant cylindrically-shaped structure, the distal end of the inner layer 40 can include an atraumatic, non-expandable distal tip 48. As illustrated in FIGS. 31, 32, and 36, the distal tip 48 defines a tubular structure with a slightly tapering or frusto-conical distal end. The outer surface of the distal tip 48 includes a distally tapering and/or curved shape such that a diameter of the distal tip 48 adjacent the tubular member 44 is greater than the diameter of the distal end of the distal tip 48. In some examples, the inner surface of the central lumen of the distal tip 48 includes a distally tapering and/or curved shape. In other examples, the central lumen of the distal tip 48 has a constant inner diameter. In some examples, the thickness of the distal tip 48 decreases between the proximal and distal ends.

The distal tip 48 is formed of a non-elastic material. Because the distal tip 48 does not expand, this prevents it from folding during retrieval of the delivery apparatus 10/prosthetic heart valve 12. In some examples, the distal tip 48 is formed from the same material as the tubular member 44. In other examples, the distal tip 48 is formed from a different elastic material as the tubular member 44. The distal tip 48 is integrally formed with the tubular member 44. For example, the distal tip 48 can be co-extruded with the tubular member 44 or reflowed from the existing tubular member 44 into the desired shape. In other examples, the distal tip 48 is separately formed and coupled to the tubular member 44. For example, the distal tip 48 can be extruded/molded separately and coupled to the tubular member 44 using heat processing. Regardless of whether integrally or separately formed with the tubular member 44, the distal tip 48 has a lower hardness/is softer than a proximal end portion of the inner layer 40. A softer tip will reduce the risk of trauma to the vessel. For example, when the delivery apparatus 10 and/or prosthetic heart valve 12 are not present in the inner layer 40, the distal tip 48 may rest against the vessel walls and abrade or otherwise damage them if it is too stiff. A softer distal tip 48, such as described here, will deflect and blunt upon interaction with the vessel wall preventing damage or trauma.

The structure of the distal tip 48 increases the structural rigidity of the distal end of the sheath assembly 22 and helps prevent buckling and/or folding of either the inner layer 40 and/or outer layer 30 during retrieval of the delivery apparatus 10/prosthetic heart valve 12. As illustrated in FIGS. 31, 32 and 36, this structure also provides a smooth, tapered profile between the inner layer 40 and the introducer 26 for pushing the inner layer 40 and introducer 26 through the central lumen of the outer layer 30.

As will be described in more detail below and as illustrated in FIGS. 34 and 36, the when the inner layer 40 is fully received within the central lumen 42 of the outer layer 30, a length of the inner layer 40 extends through and/or beyond a distal opening of the central lumen 32 of the outer layer 30. The protruding length of the inner layer 40 includes the distal tip 48. The protruding length can also include a portion of the tubular member 44 of the inner layer 40 adjacent the distal tip 48. Accordingly, the structure of the distal tip 48 blocks blood flow between the layers and provides a smooth, tapered profile for pushing the combined outer layer 30 and inner layer 40 through.

Like the expandable sheath assembly 22 of FIG. 2, the inner and outer layers 30, 40 of the expandable sheath assembly of FIG. 25 includes hydrophilic coatings and/or lubricious liners. The coatings/liners facilitate insertion into the patient's anatomy and movement between the outer and inner layers 30, 40, reducing potential damage to the sheath and patient trauma. In some embodiments, the sheath assembly 22 can include an exterior hydrophilic coating on the outer surface of the outer layer 30 to facilitate insertion of the sheath into a patient's vessel. Similarly, a hydrophilic coating can be provided on the outer surface of the inner layer 40 and/or the central lumen 32 of the outer layer 30 to reduce friction between the outer layer 30 and the inner layer 40 when the inner layer 40 is received and/or moved within the central lumen 32 of the outer layer 30. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N. V, Heerlen, the Netherlands), as well as other hydrophilic coatings (e.g., PTFE, polyethylene, polyvinylidine fluoride), are also suitable for use with the sheath assembly 22. Such hydrophilic coatings may also be included on the inner surface of the inner layer 30 to reduce friction between the sheath and the delivery apparatus 10/prosthetic heart valve 12, thereby facilitating use and improving safety. In some embodiments, a hydrophobic coating, such as Perylene, may be used on the outer surface of the outer layer 30 or the inner surface of the inner layer 40 in order to reduce friction.

In some embodiments of a sheath assembly 22 include a lubricious liner 43 on the inner surface of the inner layer 40 to reduce friction between the inner layer 40 and a passing medical device. Examples of suitable lubricious liners include materials that can further reduce the coefficient of friction of the inner layer 40, such as PTFE, polyethylene, polyvinylidine fluoride, and combinations thereof. Suitable materials for a lubricious liner also include other materials desirably having a coefficient of friction of 0.5 or less, preferably 0.1 or less.

Similar to the expandable sheath assembly 22 of FIG. 2, the outer and inner layers 30, 40 of the expandable sheath assembly of FIG. 25 are each coupled at their proximal ends to a hub 35, 45. The hub(s) can function as a handle for the expandable sheath assembly 22. Examples of such hubs is described in U.S. Provisional Patent Application No. 63/077,899 (titled “Reverse Bayonet Locking Hub,” filed Sep. 14, 2020), the disclosure of which is hereby incorporated by reference. In certain embodiments, the outer layer hub 35 can include a hemostasis valve that forms a seal around the outer surface of the guide catheter 14 once inserted through the housing to prevent leakage of pressurized blood. Similarly, the inner layer hub 45 can include a hemostasis valve that forms a seal around the outer surface of the outer layer 30 to prevent leakage of pressurized blood therebetween.

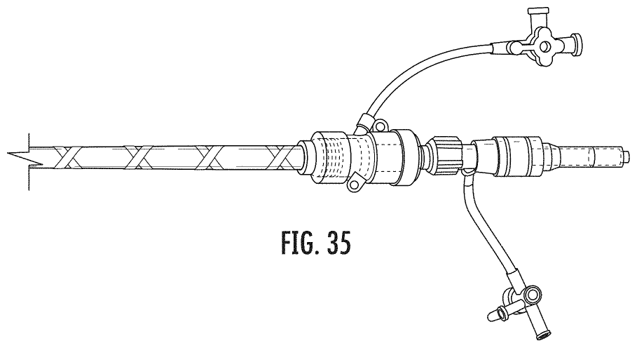

When the inner layer 40 is fully inserted within the central lumen 32 of the outer layer 30 (FIGS. 34 and 35), the inner and outer layers 40, 30 are coupled at their proximal ends. For example, hub 35 and hub 45 can include a locking or engagement feature for removably coupling when the inner layer 40 is fully inserted within the outer layer 30. When in a locked position, the locking feature fixes the axial and rotational position of the inner layer 40 with respect to the outer layer 30.

In some example sheath assembles 22, the outer and/or inner layer 30, 40 can include a radiopaque marker which can be provided to improve visibility under fluoroscopy or other similar techniques. The radiopaque marker can be located proximate the distal tip 38, 48 of the outer and/or inner layers 30, 40. Additionally or alternatively, the radiopaque marker can be provided along a length of the tubular member 34, 44 of the outer and/or inner layers 30, 40. The radiopaque marker can be in the form of a disk or tag embedded within the outer and/or inner layers 30, 40. The radiopaque marker can be a radiopaque ring provided circumferentially around the outer and/or inner layers 30, 40. The radiopaque marker can be incorporated into one or more of the axial supports 36 of the outer layer 30 and/or the reinforcing member 46 of the inner layer 40.

A method of delivering a medical device using a two-component sheath assembly 22 as illustrated in FIG. 25 is disclosed herein. The method comprises inserting an outer layer 30 of the sheath assembly 22 at least partially into the vasculature of the patient and advancing the outer layer 30 to the implantation site within the blood vessel. The outer layer 30 including a first polymeric layer and a braided layer comprising a plurality of filaments braided together. An introducer 24 is provided within the central lumen 32 of the outer layer 30 to provide column strength during insertion and positioning. The outer layer 30 is an expandable sheath movable between a non-expanded configuration and an expanded configuration. During insertion and positioning, the outer layer 30 is in the non-expanded configuration. The relative low profile and softness of the outer layer 30 enable it to easily bend along the vascular path with minimal risk of trauma.

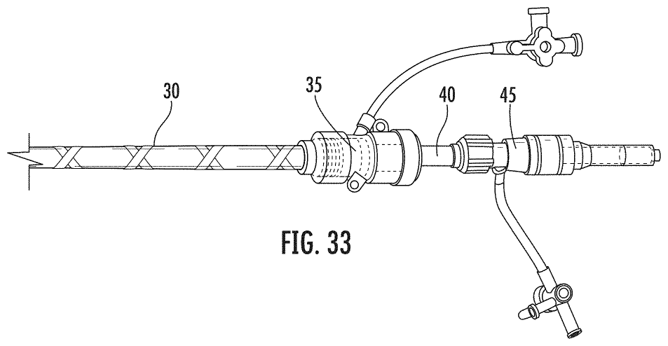

Once positioned at the treatment site, the introducer 24 is removed from the outer layer 30 and the inner layer 40 is advanced within the central lumen 32 of the outer layer 30. An introducer 26 is provided within the central lumen 32 of the inner layer 40. FIG. 33 provides a side view of the inner layer 40 partially inserted into the outer layer 30. During insertion, at least a portion of the outer layer 30 is expanding from the non-expanded configuration towards the expanded configuration by the radially outward force exerted on an inner surface of the outer layer 30 by the advancement of the inner layer 40.

The inner layer 40 is advanced fully into the outer layer 30 such that a portion of the inner layer 40 extends through and/or beyond the distal opening of the outer layer 30. FIGS. 34, 35 and 36 provide a side view of the inner layer 40 fully inserted into the outer layer 30. As illustrated in the enlarged side view of FIG. 36, the distal tip 48 of the inner layer 40 extends beyond the distal opening of the outer layer 30. In some examples, a portion of the tubular member 44 also extends beyond the distal opening of the outer layer 30. The structure of the distal tip 48 provides a smooth transition between the introducer 26 and the inner layer 40. The structure of the distal tip 38 provides a smooth transition between the inner layer 40 and the outer layer 30. This structure of the distal tips 38, 48 blocks blood flow between the layers and provides a smooth, tapered profile for pushing/positioning the combined outer layer 30 and inner layer 40 through within the patient's blood vessel.

The insertion of the inner layer 40 into the outer layer 30 gently expands the outer layer 30 to create an enlarged central lumen for the delivery apparatus 10. As described above, the outer layer 30 comprises first polymeric layer 102 and a braided second layer 104. The braided second layer 104 includes a plurality of filaments braided together. In some examples, as the outer layer 30 expands and contracts the individual filament move relative to each other to facilitate expansion of the braided second layer 104. In some examples, the braided second layer 104 is encapsulated between two polymeric layers. Because the braided second layer 104 is not adhered to the polymeric layers 102 and 108, the change in length of the braided second layer 104 that accompanies a change in the angle between the filaments does not result in a significant change in the length of the outer layer 30.

With the inner layer 40 fully inserted into the outer layer 30, the introducer 26 can be removed from the central lumen 42 of the inner layer 40.

The inner layer 40 can be coupled to the outer layer 30 such that the axial and rotational positions of each are fixedly coupled together. For example, the proximal end of the inner layer 40 is coupled with the proximal end of the outer layer 30 at the outer layer hub 35 and inner layer hub 45.

The delivery apparatus 10 and prosthetic heart valve 12 are advanced to the treatment site via the central lumen 42 of the inner layer 40. The prosthetic heart valve 12 can include a stent mounted heart valve mounted in a radially crimped state on a delivery apparatus 10. The delivery apparatus 10 and the prosthetic heart valve 12 are advanced through the central lumen 42 of the inner layer 40 and into the vasculature. The prosthetic heart valve 12 is then implanted at the treatment site within the patient. If a stent mounted heart valve is used, the heart valve is expanded after it exits the central lumen 42 of the inner layer 40.

With the prosthetic heart valve 12 implanted, the delivery apparatus 10 is removed from the central lumen 42 of the inner layer 40.

The inner layer 40 is then removed from the central lumen 32 of the outer layer 30. Because the outer layer 30 is biased to the non-expanded configuration, as the inner layer 40 is removed, the outer layer 30 locally contracts from the expanded configuration at least partially back to/towards the non-expanded configuration. As discussed above, the inner layer 40 can include a tear away feature that allows the inner layer 40 to break/spilt axially. The inner layer 40 can be removed from the central lumen 32 of the outer layer 30 by withdrawing the inner layer 40 axially (in a proximal direction) and/or engaging the tear away feature to break apart/split the inner layer 40 along its length. With the longitudinal slit/split created the inner layer 40 can be removed from the outer layer 30. It is contemplated that the inner layer 40 can be removed from the outer layer 30 before or after the prosthetic heart valve 12 is delivered to/implanted at the treatment site. With the inner layer 40 removed and the outer layer 30 remaining in place additional and/or alternate delivery apparatus and/or medical devices can be advanced through the central lumen 30 to the treatment site.

With the procedure complete, the outer layer 40 is removed from the patient's vasculature and any surgically created openings closed.

Beyond transcatheter heart valves, the expandable sheath assembly 22 can be useful for other types of minimally invasive surgery, such as any surgery requiring introduction of an apparatus into a subject's vessel. For example, the expandable sheath assembly 22 can be used to introduce other types of delivery apparatus for placing various types of intraluminal devices (e.g., stents, stented grafts, balloon catheters for angioplasty procedures, etc.) into many types of vascular and non-vascular body lumens (e.g., veins, arteries, esophagus, ducts of the biliary tree, intestine, urethra, fallopian tube, other endocrine or exocrine ducts, etc.).

### EXEMPLARY ASPECTS

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: An expandable sheath for delivering a medical device, the sheath comprising: an outer layer including an elongated tubular member with integrated axial support extending longitudinally therein, the outer layer movable between a non-expanded configuration and an expanded configuration, where the outer layer/tubular layer is biased to the non-expanded configuration; an inner layer (e.g., reinforced layer) received within a central lumen of the outer layer and movable therein, wherein receipt of the inner layer within the central lumen of the outer layer causes the outer layer to transition from the non-expanded configuration to the expanded configuration.

Example 2: The sheath according to any example here, particularly example 1, wherein insertion of the inner layer within the central lumen of the outer layer causes the outer layer to locally expand from the non-expanded configuration to the expanded configuration by a radially outward force exerted on a surface of the central lumen of the outer layer by the inner layer.

Example 3: The sheath according to any example here, particularly examples 1-2, wherein removal of the inner layer from the central lumen of the outer layer causes the outer layer to locally contract from the expanded configuration at least partially back to the non-expanded configuration.

Example 4: The sheath according to any example here, particularly examples 1-3, wherein the axial support includes a plurality of axial supports spaced circumferentially around the tubular member.

Example 5: The sheath according to any example here, particularly example 4, wherein each of the plurality of axial supports are equally spaced and/or symmetrically spaced circumferentially around the tubular member.

Example 6: The sheath according to any example here, particularly examples 1-5, wherein, when the outer layer transitions from the non-expanded configuration to the expanded configuration, the circumferential spacing between each of the plurality of axial supports increases, wherein, when the outer layer transitions from the expanded configuration to the non-expanded configuration, the circumferential spacing between each of the plurality of axial supports decreases.

Example 7: The sheath according to any example here, particularly examples 4-6, wherein the plurality of axial supports includes between 2 and 30 individual axial supports.

Example 8: The sheath according to any example here, particularly example 7, wherein the plurality of axial supports includes 20 individual axial supports.

Example 9: The sheath according to any example here, particularly examples 1-8, wherein a portion of an outer surface of the axial support protrudes from an inner surface of the central lumen of the outer layer such that the outer surface of the axial support facilitates relative movement between the outer layer and the inner layer moving within the central lumen of the outer layer.

Example 10: The sheath according to any example here, particularly example 9, wherein the axial support reduces contact area between the outer layer and the inner layer.

Example 11: The sheath according to any example here, particularly example 1-10, wherein the axial support extends along an entire length of the outer layer.

Example 12: The sheath according to any example here, particularly examples 1-10, wherein the axial support extends along a portion of an entire length of the outer layer.

Example 13: The sheath according to any example here, particularly examples 1-12, wherein the axial support has at least one of a curvilinear, rectilinear, and irregular shape in cross-section.

Example 14: The sheath according to any example here, particularly example 13, wherein the axial support has a circular shape in cross-section.

Example 15: The sheath according to any example here, particularly example 14, wherein a diameter of the axial support ranges between about 0.020 inches and about 0.005 inches, preferably about 0.010 inches.

Example 16: The sheath according to any example here, particularly examples 1-15, wherein the tubular member is composed of an elastic material.

Example 17: The sheath according to any example here, particularly examples 1-16, wherein the tubular member is composed of an elastomeric material.

Example 18: The sheath according to any example here, particularly examples 1-17, wherein the tubular member comprises a styrene-based elastomer, polyurethane, latex, copolymers thereof, blends thereof, or co-extrudates of thereof.

Example 19: The sheath according to any example here, particularly examples -1-18, wherein the tubular member is composed of a material having a 2:1 stretching ratio (e.g., the tubular member is composed of a material that has a stretched percentage of 100% unstretched compared to stretched).

Example 20: The sheath according to any example here, particularly examples 1-19, wherein an inner diameter of the outer layer in the non-expanded configuration ranges between about 8 F and about 14 F.

Example 21: The sheath according to any example here, particularly examples 1-20, wherein an inner diameter of the outer layer in the expanded configuration ranges between about 20 F and about 28 F.

Example 22: The sheath according to any example here, particularly example 21, wherein the inner diameter of the outer layer in the expanded configuration is about 24 F.

Example 23: The sheath according to any example here, particularly examples 1 22, wherein a column strength of the axial support is greater than a column strength of the tubular member.

Example 24: The sheath according to any example here, particularly examples 1-23, wherein the axial support is formed of a material with a higher rigidity than the tubular member.

Example 25: The sheath according to any example here, particularly examples 1-24, wherein the axial support is formed of a material with a higher durometer than the tubular member.

Example 26: The sheath according to any example here, particularly example 25, wherein the durometer of the axial support ranges between about 10 D and about 75 D, wherein the durometer of the tubular member ranges between about 10 A and about 45 D.

Example 27: The sheath according to any example here, particularly examples 1-26, wherein a column strength of the axial support of greater than a column strength of the inner layer.

Example 28: The sheath according to any example here, particularly examples 1-27, wherein the axial support is composed of a material that stretches/elongates less than 1% under tension.

Example 29: The sheath according to any example here, particularly examples 1-28, wherein the axial support is composed of a material that does not stretch/elongate under tension.

Example 30: The sheath according to any example here, particularly examples 1-29, wherein an overall length of the outer layer does not change when the outer layer transitions between the non-expanded and the expanded configuration.

Example 31: The sheath according to any example here, particularly examples 1-30, wherein the axial support is composed of a material comprising a high-density polyethylene, polypropylene, polyamide, fluoropolymer, copolymers thereof, or blends thereof.

Example 32: The sheath according to any example here, particularly examples 1-31, wherein the axial support is composed of a material comprising a metal, a shape memory alloy, or a combination thereof.

Example 33: The sheath according to any example here, particularly example 32, wherein the shape memory alloy comprises nitinol.

Example 34: The sheath according to any example here, particularly examples 1-33, wherein the axial support is composed of coiled wire.

Example 35: The sheath according to any example here, particularly examples 1-34, wherein the outer layer includes an atraumatic, expandable distal tip.

Example 36: The sheath according to any example here, particularly example 35, wherein the distal tip includes a distally tapering shape.

Example 37: The sheath according to any example here, particularly examples 35-36, wherein the distal tip is formed of an elastomeric material.

Example 38: The sheath according to any example here, particularly example 35-37, wherein the distal tip is formed from a same material as the tubular member.

Example 39: The sheath according to any example here, particularly examples 35-37, wherein the distal tip is formed from a different material as the tubular member.

Example 40: The sheath according to any example here, particularly examples 35-39, wherein the distal tip is integrally formed with the tubular member.

Example 41: The sheath according to any example here, particularly examples 35-39, wherein the distal tip is coupled to the tubular member.

Example 42: The sheath according to any example here, particularly examples 1-41, wherein radial expansion of the inner layer is less than 1%.

Example 43: The sheath according to any example here, particularly examples 1-42, wherein the inner layer does not expand radially.

Example 44: The sheath according to any example here, particularly examples 1-43, wherein an inner diameter of the inner layer ranges between about 16 F and about 28 F, and about 20 F and about 28 F.

Example 45: The sheath according to any example here, particularly example 44, wherein the inner diameter of the inner layer is about 24 F.

Example 46: The sheath according to any example here, particularly examples 1-45, wherein the inner layer is composed of a stiff material.

Example 47: The sheath according to any example here, particularly examples 1-46, wherein the inner layer has a column strength equal to or greater than a column strength of the outer layer.

Example 48: The sheath according to any example here, particularly examples 1-47, wherein the inner layer has a durometer ranging between about 25 D to about 75 D.

Example 49: The sheath according to any example here, particularly example 48, wherein the durometer of the inner layer varies along a longitudinal length of the inner layer.

Example 50: The sheath according to any example here, particularly examples 1-49, wherein the inner layer is more rigid than the outer layer and provides increased kink resistance upon axial load.

Example 51: The sheath according to any example here, particularly examples 1-50, wherein the inner layer is more flexible than the outer layer.

Example 52: The sheath according to any example here, particularly examples 1-51, wherein the inner layer is composed of a material comprising polytetrafluoroethylene (PTFE), expanded polytetrafluoroethylene (ePTFE), ethylene tetrafluoroethylene (ETFE), nylon, polyethylene, polyether block amide (e.g., Pebax), high density polyethylene (HDPE), and/or combinations of any of the above.

Example 53: The sheath according to any example here, particularly examples 1-52, wherein the inner layer includes a reinforcing member that extends circumferentially around the inner layer.

Example 54: The sheath according to any example here, particularly example 53, wherein the reinforcing member extends along an entire length of the inner layer.

Example 55: The sheath according to any example here, particularly example 53, wherein the reinforcing member extends along a portion of an entire length of the inner layer.

Example 56: The sheath according to any example here, particularly examples 53-55, wherein the reinforcing member maintains a diameter of the central lumen of the reinforcing member when traversing bends in the patient's vasculature and prevents kinking.

Example 57: The sheath according to any example here, particularly examples 53-56, wherein the reinforcing member is provided on an outer surface of the inner layer, on an inner surface of the central lumen of the inner layer, and/or is embedded within a tubular wall structure of the inner layer.

Example 58: The sheath according to any example here, particularly examples 53-57, wherein the reinforcing members is at least one of a coil, a braid and a mesh.

Example 59: The sheath according to any example here, particularly example 58, wherein the mesh comprises a woven mesh tube, a cut mesh tube, or a combination thereof.

Example 60: The sheath according to any example here, particularly examples 58-59, wherein the braid and/or mesh has a uniform braid density along an entire length of the reinforcing member.

Example 61: The sheath according to any example here, particularly examples 58-60, wherein the braid and/or mesh has a varying braid density along an entire length of the reinforcing member.

Example 62: The sheath according to any example here, particularly examples 58-61, wherein the braid and/or mesh has a pick count ranging between about 4 picks per inch and about 50 picks per inch.

Example 63: The sheath according to any example here, particularly examples 58-62, wherein the pick count is consistent along an entire length of the reinforcing member.

Example 64: The sheath according to any example here, particularly examples 58-62, wherein the pick count varies along an entire length of the reinforcing member.

Example 65: The sheath according to any example here, particularly example 58-64, wherein the braid and/or mesh defines a diamond-shaped pattern.

Example 66: The sheath according to any example here, particularly examples 58-65, wherein the braid and/or mesh comprises at least one filament composed of a material comprising stainless steel, nitinol, a polymer material, or a composite material.

Example 67: The sheath according to any example here, particularly examples 58-66, wherein the braid and/or mesh comprises at least one filament composed of a material comprising a polymeric material.

Example 68: The sheath according to any example here, particularly example 67, wherein the polymeric material comprises polyolefin, polyamide fiber, or combinations thereof.

Example 69: The sheath according to any example here, particularly examples 67-68, wherein the polymeric material comprises a polyester, a nylon, or a combination thereof.

Example 70: The sheath according to any example here, particularly examples 58-69, wherein the braid and/or mesh comprises at least one filament that is a round filament or a flat filament.

Example 71: The sheath according to any example here, particularly example 70, wherein the round filament has a diameter ranging between 0.001 inches and about 0.015 inches.

Example 72: The sheath according to any example here, particularly example 70, wherein the flat filament has a height ranging between 0.001 inches and 0.015 inches and a width ranging between about 0.001 inches and about 0.015 inches.

Example 73: The sheath according to any example here, particularly examples 58-72, wherein the coil is wound to resist axial compression applied to the inner layer, while facilitating bending of the inner layer in a direction away from the longitudinal axis of the inner layer.

Example 74: The sheath according to any example here, particularly examples 58-73, wherein the coil includes a coil winding which provides compressive stiffness to the inner layer during axial compressive loads.

Example 75: The sheath according to any example here, particularly example 74, wherein the coil winding extends helically about a longitudinal axis of the inner layer and defines a central lumen between its proximal and distal ends.

Example 76: The sheath according to any example here, particularly examples 74-75, wherein the coil has a plurality of tightly wound turns of the coil winding.

Example 77: The sheath according to any example here, particularly examples 74-76, wherein the coil winding has a constant pitch along an axial length of the coil.

Example 78: The sheath according to any example here, particularly examples 74-77, wherein the coil winding has a varying pitch along an entire length of the coil winding/coil.

Example 79: The sheath according to any example here, particularly example 74-78, wherein a pitch of the coil winding at a distal portion of the coil can be less than a pitch of the coil winding at a proximal portion of the coil.

Example 80: The sheath according to any example here, particularly examples 74-79, wherein the coil winding has a pitch angle ranging between about 10 degrees and about 180 degrees.

Example 81: The sheath according to any example here, particularly examples 74-80, wherein a gap/spacing is provided between adjacent turns of the coil winding.

Example 82: The sheath according to any example here, particularly example 81, wherein the coil is tightly wound such that the adjacent turns of the coil winding contact.

Example 83: The sheath according to any example here, particularly examples 74-82, wherein the coil winding has a pitch ranging between 1 turns per inch and 40 turns per inch.

Example 84: The sheath according to any example here, particularly example 83, wherein the pitch ranges between 30 turns per inch and 40 turns per inch.

Example 85: The sheath according to any example here, particularly examples 74-84, wherein the coil winding has a diameter ranging between 0.001 inches and about 0.015 inches.

Example 86: The sheath according to any example here, particularly examples 74-85, wherein the coil winding is composed of a material comprising stainless steel, nitinol, a polymer material, or a composite material.

Example 87: The sheath according to any example here, particularly examples 1-86, wherein the inner layer includes an atraumatic, non-expandable distal tip.

Example 88: The sheath according to any example here, particularly example 87, wherein when the inner layer is fully received within the central lumen of the outer layer, a length of the inner layer extends through and/or beyond a distal opening of the central lumen of the outer layer.

Example 89: The sheath according to any example here, particularly example 88, wherein the length of the inner layer includes the distal tip.

Example 90: The sheath according to any example here, particularly example 89, wherein the length of the inner layer includes a portion of the tubular member of the inner layer adjacent the distal tip.

Example 91: The sheath according to any example here, particularly examples 87-90, wherein the distal tip includes a distally tapering shape.

Example 92: The sheath according to any example here, particularly examples 87-91, wherein the distal tip is formed from a non-elastic material.

Example 93: The sheath according to any example here, particularly examples 87-92, wherein the distal tip is formed from a same material as the inner layer.

Example 94: The sheath according to any example here, particularly examples 87-92, wherein the distal tip is formed from a different material as the inner layer.

Example 95: The sheath according to any example here, particularly examples 87-94, wherein the distal tip has a lower hardness than a proximal end portion of the inner layer.

Example 96: The sheath according to any example here, particularly examples 87-95, wherein the distal tip is integrally formed with the inner layer.

Example 97: The sheath according to any example here, particularly examples 87-95, wherein the distal tip is coupled to the inner layer.

Example 98: The sheath according to any example here, particularly examples 1-97, wherein the inner layer includes a hydrophilic coating on an outer surface to reduce friction between the outer layer and the inner layer when the inner layer is received and/or moved within the central lumen of the outer layer.

Example 99: The sheath according to any example here, particularly examples 1-98, wherein a central lumen of the inner layer includes a lubricious liner and/or coating to reduce friction between the inner layer and a passing medical device.

Example 100: The sheath according to any example here, particularly example 99, wherein the lubricious liner and/or coating is composed of a material comprising polytetrafluoroethylene (PTFE), polyethylene, polyvinylidine fluoride, and combinations thereof.

Example 101: The sheath according to any example here, particularly examples 99-100, wherein the lubricous liner and/or coating is composed of a material desirably having a coefficient of friction of 0.5 or less.

Example 102: The sheath according to any example here, particularly examples 99-101, wherein the lubricous liner and/or coating is composed of a material desirably having a coefficient of friction of 0.1 or less.

Example 103: The sheath according to any example here, particularly examples 1-102, wherein the central lumen of the outer layer includes a hydrophilic coating to reduce friction between the outer layer and the inner layer when the inner layer is received and/or moved within the central lumen of the outer layer.

Example 104: The sheath according to any example here, particularly examples 1-103, wherein an outer surface of the outer layer includes a hydrophilic coating to reduce friction between the outer layer and patient's vasculature.

Example 105: The sheath according to any example here, particularly examples 1-104, wherein a proximal end of the outer layer is coupled to a hub having a proximal end and a distal end and defining a central lumen extending longitudinally between the proximal and distal end.

Example 106: The sheath according to any example here, particularly example 105, wherein the central lumen of the hub includes a seal for preventing unwanted fluid from advancing in a proximal and/or distal direction within the central lumen of the hub.

Example 107: The sheath according to any example here, particularly examples 1-106, wherein a proximal end of the inner layer is coupled to an inner layer hub having a proximal end and a distal end and defining a central lumen extending longitudinally between the proximal and distal end, wherein a proximal end of the outer layer is coupled to an outer layer hub having a proximal end and a distal end and defining a central lumen extending longitudinally between the proximal and distal end.

Example 108: The sheath according to any example here, particularly example 107, wherein the central lumen of the inner layer hub and the outer layer hub each includes a seal for preventing unwanted fluid from advancing in a proximal and/or distal direction within the central lumen of the respective hub.

Example 109: The sheath according to any example here, particularly examples 1-108, wherein the outer layer and the inner layer are coupled together at the proximal end when the inner layer is fully received within the central lumen of the outer layer.

Example 110: The sheath according to any example here, particularly example 109, wherein a proximal end of the outer layer is coupled to an outer layer hub, wherein a proximal end of the inner layer is coupled to an inner layer hub, wherein the outer layer hub and the inner sheath hub are removably coupled when the inner layer is fully received within the central lumen of the outer layer.

Example 111: The sheath according to any example here, particularly examples 1-110, further comprising a radiopaque marker provided in at least one the outer layer and the inner layer.

Example 112: The sheath according to any example here, particularly example 111, wherein the radiopaque marker is located proximate a distal tip of the outer layer.

Example 113: The sheath according to any example here, particularly examples 111-112, wherein the radiopaque marker is located proximate a distal tip of the inner layer.

Example 114: A method of delivering a prosthetic device into a patient, the method comprising: inserting an outer layer at an implantation site within a blood vessel of the patient, the outer layer including an elongated tubular member with integrated axial supports extending longitudinally therein, an introducer extending within a central lumen of the outer layer, where the outer layer movable between a non-expanded configuration and an expanded configuration, where the outer layer is biased to the non-expanded configuration; removing the introducer from the central lumen of the outer layer; advancing an inner layer within the central lumen of the outer layer, an introducer extending within a central lumen of the inner layer; locally expanding a portion of the outer layer from the non-expanded configuration towards the expanded configuration by a radially outward force exerted on an inner surface of the outer layer by the advancement of the inner layer; removing the introducer from within the central lumen of the inner layer; advancing a prosthetic delivery device through a central lumen of the inner layer; delivering a prosthetic delivery device to a treatment site; removing the prosthetic delivery device from the central lumen of the inner layer; removing the inner layer from the central lumen of the outer layer; and locally contracting the portion of the outer layer from the expanded configuration at least partially back to the non-expanded configuration upon removal of the inner layer from the portion of the outer layer.

Example 115: The method according to any example here, particularly example 114, wherein the axial supports are spaced circumferentially around the elongated tubular member, wherein locally expanding a portion of the outer layer to/towards the expanded configuration increases a circumferential spacing between the axial supports.

Example 116: The method according to any example here, particularly examples 114-115, wherein locally contracting the portion of the outer layer to/toward the non-expanded configuration decreases a circumferential spacing between the axial supports.

Example 117: The method according to any example here, particularly examples 114-116, wherein a portion of an outer surface of the axial support protrudes from an inner surface of the central lumen of the outer layer, wherein the outer surface of the axial support provides a bearing surface between the outer layer and the inner layer when the inner layer is advanced within and/or removed from the central lumen of the outer layer.

Example 118: The method according to any example here, particularly examples 114-117, further comprising coupling a proximal end of the inner layer with a proximal end of the outer layer when the inner layer is fully advanced within the central lumen of the outer layer.

Example 119: The method according to any example here, particularly examples 114-118, wherein the prosthetic device is a stent mounted heart valve mounted in a radially crimped state on a delivery apparatus, wherein advancing the prosthetic device through the central lumen of the inner layer comprises advancing the prosthetic delivery device and the heart valve through the central lumen of the inner layer and into the vasculature, wherein delivering the prosthetic device includes implanting the heart valve at the treatment site within the patient.

Example 120: The method according to any example here, particularly examples 114-119, wherein implanting the prosthetic device at the treatment site further comprises, expanding the stent-mounted heart valve after it exits the central lumen of the inner layer.

Example 121: A delivery catheter assembly comprising: a proximal region comprising a hub with a hemostasis valve; the expandable sheath of any one of claims 1-113, the outer layer coupled to and extending distally from the hub and fluidically coupled to the hemostasis valve; a guide catheter slidably positionable within a central lumen of the inner layer; a balloon catheter positionable within the guide catheter, a distal region of the balloon catheter comprising an inflatable balloon; an implantable device configured to be coupled to the inflatable balloon, and a capsule configured to extend over the implantable device.

Example 122: The assembly according to any example here, particularly example 121, wherein the implantable device is a heart valve.

Example 123: The assembly according to any example here, particularly examples 121 and 122, wherein the guide catheter is steerable.

Example 124: The assembly according to any example here, particularly examples 121-123, further comprising a nose cone at the distal region of the balloon catheter.

Example 125: The assembly according to any example here, particularly examples 121-124, wherein the hemostasis valve is housed within the hub.

Example 126: The assembly according to any example here, particularly examples 121-125, wherein the proximal region of the delivery catheter assembly further comprises a handle.

Example 127: The assembly according to any example here, particularly examples 121-126, wherein the handle further comprises an infusion port.

Example 128: An expandable sheath for delivering a medical device, the sheath comprising: an outer layer including a first polymeric layer and a braided layer comprising a plurality of filaments braided together, the outer layer movable between a non-expanded configuration and an expanded configuration, where the outer layer/tubular layer is biased to the non-expanded configuration; an inner layer received within a central lumen of the outer layer and movable therein, wherein receipt of the inner layer within the central lumen of the outer layer causes the outer layer to transition from the non-expanded configuration to the expanded configuration.

Example 129: The sheath according to any example here, particularly example 128, wherein insertion of the inner layer within the central lumen of the outer layer causes the outer layer to locally expand from the non-expanded configuration to the expanded configuration by a radially outward force exerted on a surface of the central lumen of the outer layer by the inner layer.

Example 130: The sheath according to any example here, particularly example 129, wherein removal of the inner layer from the central lumen of the outer layer causes the outer layer to locally contract from the expanded configuration at least partially back to the non-expanded configuration.

Example 131: The sheath according to any example here, particularly examples 128-130, wherein the outer layer further includes a second polymeric layer, wherein the braided layer is radially outward of the first polymeric layer, wherein the second polymeric layer is radially outward of the braided layer and bonded to the first polymeric layer such that the braided layer is encapsulated between the first and the second polymeric layers.

Example 132: The sheath according to any example here, particularly example 131, further comprising a resilient elastic layer radially outward of the braided layer, the elastic layer being configured to apply radial force to the braided layer and the first polymeric layer, wherein the outer layer resiliently returns toward the non-expanded configuration by radial force applied by the elastic layer upon passage of the inner layer.

Example 133: The sheath according to any example here, particularly examples 131-132, wherein when the inner layer is passed through/into the central lumen of the outer layer, a diameter of the outer layer expands from a first diameter to a second, larger, diameter around the inner layer while the first and second polymeric layers resist axial elongation of the sheath such that a length of the sheath remains substantially constant.

Example 134: The sheath according to any example here, particularly example 133, wherein the outer layer stretches/elongates less than 1% under tension.

Example 135: The sheath according to any example here, particularly examples 131-134, wherein the first and second polymeric layers comprise a plurality of longitudinally-extending folds when the sheath is in the non-expanded configuration.

Example 136: The sheath according to any example here, particularly example 135, wherein the longitudinally extending folds create a plurality of circumferentially spaced ridges and a plurality of circumferentially spaced valleys.

Example 137: The sheath according to any example here, particularly examples 135-136, wherein, as the inner layer is passed through the outer layer, the ridges and valleys level out to allow the outer layer to radially expand.

Example 138: The sheath according to any example here, particularly examples 135-137, wherein the filaments of the braided layer are movable between the first and second polymeric layers such that the braided layer is configured to radially expand as the inner layer is passed through the sheath while the length of the sheath remains substantially constant.

Example 139: The sheath according to any example here, particularly example 138, wherein the filaments of the braided layer are not engaged or adhered to the first or second polymeric layers.

Example 140: The sheath according to any example here, particularly examples 138-139, wherein the filaments of the braided layer are resiliently buckled when the outer layer is in the nonexpanded configuration.

Example 141: The sheath according to any example here, particularly example 140, wherein the first and second polymeric layers are attached to each other at a plurality of open spaces between the filaments of the braided layer.

Example 142: The sheath according to any example here, particularly examples 128-141, wherein the braided layer comprises a self-contracting material.

Example 143: The sheath according to any example here, particularly examples 128-142, wherein the braided layer comprises a shape-memory material biased toward the non-expanded configuration.

Example 144: The sheath according to any example here, particularly examples 131-143, wherein the first and second polymeric layers are composed of an elastic material.

Example 145: The sheath according to any example here, particularly examples 131-144, wherein the first and second polymeric layers are composed of an elastomeric material.

Example 146: The sheath according to any example here, particularly examples 131-145, wherein the first and second polymeric layers are composed of a material comprising ultra-high-molecular-weight polyethylene (UHMWPE) (e.g., Dyneema®), high-density polyethylene (HDPE), or polyether ether ketone (PEEK), or combinations thereof.

Example 147: The sheath according to any example here, particularly examples 131-146, wherein the first and second polymeric layers are composed of a material comprising polytetrafluoroethylene (PTFE), expanded polytetrafluoroethylene (ePTFE), ethylene tetrafluoroethylene (ETFE), nylon, polyethylene, polyether block amide (e.g., Pebax), or combinations thereof.

Example 148: The sheath according to any example here, particularly examples 131-147, wherein the first polymeric layer is composed of material having a low coefficient of friction material to facilitate passage of the inner layer through the central lumen of the outer layer.

Example 149: The sheath according to any example here, particularly examples 128-148, wherein the tubular member is composed of a material having a 2:1 stretching ratio (e.g., the tubular member is composed of a material that has a stretched percentage of 100% unstretched compared to stretched).

Example 150: The sheath according to any example here, particularly examples 128-149, wherein an inner diameter of the outer layer in the non-expanded configuration ranges from about 8 F to about 14 Fr.

Example 151: The sheath according to any example here, particularly examples 128-150, wherein an inner diameter of the outer layer in the expanded configuration ranges from about 14 Fr to about 24 Fr.

Example 152: The sheath according to any example here, particularly example 151, wherein the inner diameter of the outer layer in the expanded configuration is about 24 F

Example 153: The sheath according to any example here, particularly examples 128-152, wherein a column strength outer layer is less than a column strength of the inner layer.

Example 154: The sheath according to any example here, particularly examples 128-153, wherein the inner layer includes one or more tear away features for causing the inner layer to break or split so that it can be separated from the inner layer.

Example 155: The sheath according to any example here, particularly example 154, wherein the tear away feature includes one or more longitudinally extending slits, weakened portions, scorelines, or pull wire or combinations thereof.

Example 156: The sheath according to any example here, particularly example 155, wherein the pull wire is embedded in the inner layer.

Example 157: The sheath according to any example here, particularly examples 154-156, wherein the tear away feature extends from a proximal end of the inner layer toward the distal end.

Example 158: The sheath according to any example here, particularly examples 154-157, wherein the tear away feature extends along a portion of an entire length of the inner layer.

Example 159: The sheath according to any example here, particularly examples 154-158, wherein the tear away feature extends along an entire length of the inner layer.

Example 160: The sheath according to any example here, particularly examples 128-159, wherein the outer layer includes an atraumatic, expandable distal tip.

Example 161: The sheath according to any example here, particularly example 160, wherein the distal tip includes a distally tapering shape.

Example 162: The sheath according to any example here, particularly examples 160-161, wherein the distal tip is formed of an elastomeric material.

Example 163: The sheath according to any example here, particularly examples 160-162, wherein the distal tip is formed from a same material as the outer layer.

Example 164: The sheath according to any example here, particularly examples 160-162, wherein the distal tip is formed from a different material as the outer layer.

Example 165: The sheath according to any example here, particularly examples 160-164, wherein the distal tip is integrally formed with the outer layer.

Example 166: The sheath according to any example here, particularly examples 160-165, wherein the distal tip is coupled to the outer layer.

Example 167: The sheath according to any example here, particularly examples 128-166, wherein the inner layer does not expand radially.

Example 168: The sheath according to any example here, particularly examples 128-167, wherein an inner diameter of the inner layer ranges between about 20 F and about 28 F.

Example 169: The sheath according to any example here, particularly example 168, wherein the inner diameter of the inner layer is about 24 F.

Example 170: The sheath according to any example here, particularly examples 128-169, wherein the inner layer is composed of a stiff material.

Example 171: The sheath according to any example here, particularly examples 128-170, wherein the inner layer is composed of a material comprising polytetrafluoroethylene (PTFE), expanded polytetrafluoroethylene (ePTFE), ethylene tetrafluoroethylene (ETFE), nylon, polyethylene, polyethylene terephthalate (PET), polyether block amide (e.g., Pebax), high density polyethylene (HDPE), polyether ether ketone (PEEK), ultra-high-molecular-weight polyethylene (UHMWPE), nylon, fluorinated ethylene propylene (FEP), polypropylene (PP), and/or combinations of any of the above.

Example 172: The sheath according to any example here, particularly examples 128-171, wherein the inner layer has a column strength greater than a column strength of the outer layer.

Example 173: The sheath according to any example here, particularly examples 128-172, wherein the inner layer has a lower rigidity than the outer layer.

Example 174: The sheath according to any example here, particularly examples 128-173, wherein the inner layer is more flexible than the outer layer.

Example 175: The sheath according to any example here, particularly examples 128-174, wherein the inner layer includes an atraumatic, non-expandable distal tip.

Example 176: The sheath according to any example here, particularly example 175, wherein when the inner layer is fulling received within the central lumen of the outer layer, a distal end of the inner layer extends through and/or beyond a distal opening of the central lumen of the outer layer.

Example 177: The sheath according to any example here, particularly examples 175-176, wherein the distal tip includes a distally tapering shape.

Example 178: The sheath according to any example here, particularly examples 175-177, wherein the distal tip is formed from a same material as the inner layer.

Example 179: The sheath according to any example here, particularly examples 175-177, wherein the distal tip is formed from a different material as the inner layer.

Example 180: The sheath according to any example here, particularly examples 175-179, wherein the distal tip has a lower hardness than a proximal end portion of the inner layer.

Example 181: The sheath according to any example here, particularly examples 175-180, wherein the distal tip is integrally formed with the inner layer.

Example 182: The sheath according to any example here, particularly examples 175-180, wherein the distal tip is coupled to the inner layer.

Example 183: The sheath according to any example here, particularly examples 128-182, wherein the inner layer includes a hydrophilic coating on an outer surface to reduce friction between the inner layer and the outer layer when the inner layer is received and/or moved within the central lumen of the outer layer.

Example 184: The sheath according to any example here, particularly examples 128-183, wherein a central lumen of the inner layer includes a lubricious liner and/or coating to reduce friction between the inner layer and a passing medical device.

Example 185: The sheath according to any example here, particularly example 184, wherein the lubricious liner and/or coating is composed of a material comprising polytetrafluoroethylene (PTFE), polyethylene, polyvinylidine fluoride, and combinations thereof.

Example 186: The sheath according to any example here, particularly examples 184-185, wherein the lubricous liner and/or coating is composed of a material desirably having a coefficient of friction of 0.5 or less.

Example 187: The sheath according to any example here, particularly examples 184-186, wherein the lubricous liner and/or coating is composed of a material desirably having a coefficient of friction of 0.1 or less.

Example 188: The sheath according to any example here, particularly examples 128-187, wherein the central lumen of the outer layer includes a hydrophilic coating to reduce friction between the outer layer and the inner layer when the inner layer is received and/or moved within the central lumen of the outer layer.

Example 189: The sheath according to any example here, particularly examples 128-188, wherein an outer surface of the outer layer includes a hydrophilic coating to reduce friction between the outer layer and patient's vasculature.

Example 190: The sheath according to any example here, particularly examples 128-189, wherein a proximal end of the outer layer is coupled to a hub having a proximal end and a distal end and defining a central lumen extending longitudinally between the proximal and distal end.

Example 191: The sheath according to any example here, particularly example 190, wherein the central lumen of the hub includes a seal for preventing unwanted fluid from advancing in a proximal and/or distal direction within the central lumen of the hub.

Example 192: The sheath according to any example here, particularly examples 128-191, wherein a proximal end of the inner layer is coupled to a hub having a proximal end and a distal end and defining a central lumen extending longitudinally between the proximal and distal end.

Example 193: The sheath according to any example here, particularly example 192, wherein the central lumen of the hub includes a seal for preventing unwanted fluid from advancing in a proximal and/or distal direction within the central lumen of the hub.

Example 194: The sheath according to any example here, particularly examples 128-193, wherein the outer layer and the inner layer are coupled at their proximal end when the inner layer is fully received within the central lumen of the outer layer.

Example 195: The sheath according to any example here, particularly example 194, wherein a proximal end of the outer layer is coupled to an outer layer hub, wherein a proximal end of the inner layer is coupled to an inner sheath hub, wherein the outer layer hub and the inner sheath hub are removably coupled when the inner layer is fully received within the central lumen of the outer layer.

Example 196: The sheath according to any example here, particularly examples 128-195, further comprising a radiopaque marker provided in at least one the outer layer and the inner layer.

Example 197: The sheath according to any example here, particularly example 196, wherein the radiopaque marker is located proximate a distal tip of the outer layer.

Example 198: The sheath according to any example here, particularly examples 196-197, wherein the radiopaque marker is located proximate a distal tip of the inner layer.

Example 199: A method of delivering a prosthetic device into a patient, the method comprising: inserting an outer layer at an implantation site within a blood vessel of the patient, the outer layer including an a first polymeric layer and a braided layer comprising a plurality of filaments braided together, an introducer extending within a central lumen of the outer layer, where the outer layer movable between a non-expanded configuration and an expanded configuration, where the outer layer is biased to the non-expanded configuration; removing the introducer from the central lumen of the outer layer; advancing an inner layer within the central lumen of the outer layer, an introducer extending within a central lumen of the inner layer; locally expanding a portion of the outer layer from the non-expanded configuration towards the expanded configuration by a radially outward force exerted on an inner surface of the outer layer by the advancement of the inner layer; removing the introducer from within the central lumen of the outer layer; advancing a prosthetic delivery device through a central lumen of the inner layer; delivering a prosthetic device to a treatment site; removing the prosthetic delivery device from the central lumen of the inner layer; removing the inner layer from the central lumen of the outer layer; locally contracting the portion of the outer layer from the expanded configuration at least partially back to the non-expanded configuration upon removal of the inner layer from the portion of the outer layer; and removing the outer layer from patient's blood vessel.

Example 200: The method according to any example here, particularly example 199, further comprising coupling a proximal end of the inner layer with a proximal end of the outer layer when the inner layer is fully advanced within the central lumen of the outer layer.

Example 201: The method according to any example here, particularly examples 199-200, wherein the prosthetic device is a stent mounted heart valve mounted in a radially crimped state on a delivery apparatus, wherein advancing the prosthetic device through the central lumen of the inner layer comprises advancing the prosthetic delivery device and the heart valve through the central lumen of the inner layer and into the vasculature, wherein delivering the prosthetic devices includes implanting the heart valve at the treatment site within the patient.

Example 202: The method according to any example here, particularly examples 199-201, wherein implanting the prosthetic device at the treatment site further comprises expanding the stent-mounted heart valve after it exits the central lumen of the inner layer

Example 203: A delivery catheter assembly for delivering a medical device, the assembly comprising: a proximal region comprising a hub with a hemostasis valve; the expandable sheath of any one of claims 128-198, the outer layer coupled to and extending distally from the hub and fluidically coupled to the hemostasis valve; a guide catheter slidably positionable within a central lumen of the inner layer; a balloon catheter positionable within the guide catheter, a distal region of the balloon catheter comprising an inflatable balloon; an implantable device configured to be coupled to the inflatable balloon, and a capsule configured to extend over the implantable device.

Example 204: The delivery catheter assembly according to any example here, particularly example 203, wherein the implantable device is a heart valve.

Example 205: The delivery catheter assembly according to any example here, particularly examples 203 and/or 204, wherein the guide catheter is steerable.

Example 206: The delivery catheter assembly according to any example here, particularly examples 203-205, further comprising a nose cone at the distal region of the balloon catheter.

Example 207: The delivery catheter assembly according to any example here, particularly examples 203-206, wherein the hemostasis valve is housed within the hub.

Example 208: The delivery catheter assembly according to any example here, particularly examples 203-207, wherein the proximal region of the delivery catheter assembly further comprises a handle.

Example 209: The delivery catheter assembly according to any example here, particularly examples 203-208, wherein the handle further comprises an infusion port.

Although the foregoing embodiments of the present disclosure have been described in some detail by way of illustration and example for purposes of clarity and understanding, it will be apparent to those skilled in the art that certain changes and modifications may be practiced within the spirit and scope of the present disclosure. It is intended that the scope of the present disclosure herein disclosed should not be limited by the particular disclosed embodiments described above, but should be determined only by a fair reading of the claims that follow.

1. An expandable sheath comprising:
an outer layer including an elongated tubular member with integrated axial support extending longitudinally therein, the outer layer movable between a non-expanded configuration and an expanded configuration, where the outer layer is biased to the non-expanded configuration and the axial support includes a plurality of axial supports spaced circumferentially around the tubular member; and
an inner layer received within a central lumen of the outer layer and movable therein,
wherein receipt of the inner layer within the central lumen of the outer layer causes the outer layer to transition from the non-expanded configuration to the expanded configuration.

2. The expandable sheath of claim 1, wherein insertion of the inner layer within the central lumen of the outer layer causes the outer layer to locally expand from the non-expanded configuration to the expanded configuration by a radially outward force exerted on a surface of the central lumen of the outer layer by the inner layer,
wherein removal of the inner layer from the central lumen of the outer layer causes the outer layer to locally contract from the expanded configuration at least partially back to the non-expanded configuration,
wherein, when the outer layer transitions from the non-expanded configuration to the expanded configuration, a circumferential spacing between each of the plurality of axial supports increases,
wherein, when the outer layer transitions from the expanded configuration to the non-expanded configuration, a circumferential spacing between each of the plurality of axial supports decreases.

3. The expandable sheath of claim 1, wherein the tubular member is composed of an elastic material,
wherein a column strength of at least one of the plurality of axial supports is greater than a column strength of the tubular member, and
wherein a column strength of the inner layer is equal to or greater than a column strength of the outer layer.

4. The expandable sheath of claim 1, wherein at least one of the plurality of axial supports is composed of a material that stretches/elongates less than 1% under tension,
wherein an overall length of the outer layer does not change when the outer layer transitions between the non-expanded and the expanded configuration.

5. The expandable sheath of claim 1, wherein a portion of an outer surface of at least one of the plurality of axial supports protrudes from an inner surface of the central lumen of the outer layer such that the outer surface of the axial support facilitates relative movement between the outer layer and the inner layer moving within the central lumen of the outer layer.

6. The expandable sheath of claim 1, wherein the inner layer includes a reinforcing member that extends circumferentially around the inner layer, wherein the reinforcing member maintains a diameter of the central lumen of the reinforcing member when traversing bends in a patient's vasculature and prevents kinking.

7. The expandable sheath of claim 6, wherein the reinforcing members is at least one of a coil, a braid, or a mesh,
wherein the coil is wound to resist axial compression applied to the inner layer, while facilitating bending of the inner layer in a direction away from a longitudinal axis of the inner layer, the coil including a coil winding which provides compressive stiffness to the inner layer during axial compressive loads, the coil winding extending helically about a longitudinal axis of the inner layer and defines a central lumen between its proximal and distal ends.

8. The expandable sheath of claim 7, wherein the coil winding has a varying pitch along an entire length of the coil winding,
wherein a pitch of the coil winding at a distal portion of the coil can be less than a pitch of the coil winding at a proximal portion of the coil.

9. The expandable sheath of claim 1, wherein when the inner layer is fully received within the central lumen of the outer layer, a length of the inner layer extends through and/or beyond a distal opening of the central lumen of the outer layer.

10. The expandable sheath of claim 1, wherein the outer layer and the inner layer are coupled together at a proximal end of the inner layer when the inner layer is fully received within the central lumen of the outer layer.

11. The expandable sheath of claim 1, wherein a proximal end of the inner layer is coupled to an inner layer hub having a proximal end and a distal end and defining a central lumen extending longitudinally between the proximal and distal end,
wherein a proximal end of the outer layer is coupled to an outer layer hub having a proximal end and a distal end and defining a central lumen extending longitudinally between the proximal and distal end,
wherein the central lumen of the inner layer hub and the outer layer hub each includes a seal for preventing unwanted fluid from advancing in a proximal and/or distal direction within the central lumen of the respective hub,
wherein the outer layer hub and the inner sheath hub are removably coupled when the inner layer is fully received within the central lumen of the outer layer.

12. The expandable sheath of claim 1, further including:
a delivery catheter assembly comprising:
a proximal region comprising a hub with a hemostasis valve, where the outer layer of the expandable sheath is coupled to and extending distally from the hub and fluidically coupled to the hemostasis valve;
a guide catheter slidably positionable within the central lumen of the inner layer;
a balloon catheter positionable within the guide catheter, a distal region of the balloon catheter comprising an inflatable balloon;
an implantable device configured to be coupled to the inflatable balloon, and
a capsule configured to extend over the implantable device.

13. A method of delivering a prosthetic device into a patient, the method comprising:
inserting an outer layer at an implantation site within a blood vessel of the patient, the outer layer including an elongated tubular member with integrated axial supports extending longitudinally therein, an introducer extending within a central lumen of the outer layer, where the outer layer movable between a non-expanded configuration and an expanded configuration, where the outer layer is biased to the non-expanded configuration;
removing the introducer from the central lumen of the outer layer;
advancing an inner layer within the central lumen of the outer layer, an introducer extending within a central lumen of the inner layer;
locally expanding a portion of the outer layer from the non-expanded configuration towards the expanded configuration by a radially outward force exerted on an inner surface of the outer layer by the advancement of the inner layer;
coupling a proximal end of the inner layer with a proximal end of the outer layer when the inner layer is fully advanced within the central lumen of the outer layer;
removing the introducer from within the central lumen of the inner layer;
advancing a prosthetic delivery device through a central lumen of the inner layer;
delivering a prosthetic delivery device to a treatment site;
removing the prosthetic delivery device from the central lumen of the inner layer;
removing the inner layer from the central lumen of the outer layer; and
locally contracting the portion of the outer layer from the expanded configuration at least partially back to the non-expanded configuration upon removal of the inner layer from the portion of the outer layer.

14. The method of claim 13, wherein the axial supports are spaced circumferentially around the elongated tubular member,
wherein locally expanding a portion of the outer layer to/towards the expanded configuration increases a circumferential spacing between the axial supports,
wherein locally contracting the portion of the outer layer to/toward the non-expanded configuration decreases a circumferential spacing between the axial supports.

15. An expandable sheath comprising:
an outer layer including a first polymeric layer and a braided layer comprising a plurality of filaments braided together, the outer layer movable between a non-expanded configuration and an expanded configuration, where the outer layer is biased to the non-expanded configuration; and
an inner layer received within a central lumen of the outer layer and movable therein,
wherein receipt of the inner layer within the central lumen of the outer layer causes the outer layer to transition from the non-expanded configuration to the expanded configuration,
wherein when the inner layer is passed into the central lumen of the outer layer, a diameter of the outer layer expands from a first diameter to a second, larger, diameter around the inner layer while the first and second polymeric layers resist axial elongation of the sheath such that a length of the sheath remains substantially constant.

16. The expandable sheath of claim 15, wherein the outer layer further includes a second polymeric layer,
wherein the braided layer is radially outward of the first polymeric layer,
wherein the second polymeric layer is radially outward of the braided layer and bonded to the first polymeric layer such that the braided layer is encapsulated between the first and the second polymeric layers.

17. The expandable sheath of claim 16, further comprising a resilient elastic layer radially outward of the braided layer, the elastic layer being configured to apply radial force to the braided layer and the first polymeric layer,
wherein the outer layer resiliently returns toward the non-expanded configuration by radial force applied by the elastic layer upon passage of the inner layer.

18. The expandable sheath of claim 15, wherein the inner layer includes one or more tear away features for causing the inner layer to break or split so that it can be separated from the inner layer, the tear away feature extends from a proximal end of the inner layer toward a distal end and includes one or more longitudinally extending slits, weakened portions, scorelines, or pull wire or combinations thereof.

19. The expandable sheath of claim 15, wherein the inner layer includes an atraumatic, non-expandable distal tip, wherein when the inner layer is fulling received within the central lumen of the outer layer, a distal end of the inner layer extends through a distal opening of the central lumen of the outer layer.

20. The expandable sheath of claim 15, wherein a proximal end of the outer layer is coupled to an outer layer hub,
wherein a proximal end of the inner layer is coupled to an inner sheath hub,
wherein the outer layer hub and the inner sheath hub are removably coupled when the inner layer is fully received within the central lumen of the outer layer.
